(19) United States
(12) Patent Application Publication
(10) Pub. No.: US 2007/0059247 A1
(43) Pub. Date: Mar. 15, 2007

(54) Deposit contrast agents and related methods thereof

(76) Inventors: Jonathan R. Lindner, Portland, OR (US); Beat A. Kaufmann, Portland, OR (US); Alexander L. Klibanov, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/511,721

(22) Filed: Aug. 29, 2006

Related U.S. Application Data
(60) Provisional application No. 60/712,657, filed on Aug. 30, 2005.
(60) Provisional application No. 60/735,517, filed on Nov. 11, 2005.

(51) Int. Cl. A61K 49/22

(52) U.S. Cl. 424/9.52

(57) ABSTRACT

A method for generating an enhanced ultrasound image comprises intravenously administering to a subject a plurality of microbubbles of sufficient diameter to lodge in the microvasculature of a subject. An ultrasound image of a portion of the subject is generated wherein the image is enhanced by one or more of the administered microbubbles that has lodged in the microvasculature of the imaged portion. An ultrasound contrast media composition comprises a plurality of gas filled microbubbles. At least about 5% of the microbubbles have a diameter of at least about 4 microns (μm), and wherein the composition is suitable for intravenous administration. The administered microbubbles are of sufficient diameter to lodge in the microvasculature of a subject and can be used enhance ultrasound images small animal subjects including mice, rats and rabbits. The described methods and compositions can be used to enhance ultrasound images produced using high frequency ultrasound.

### CROSS-REFERENCE TO RELATED APPLICATIONS

This application claims the benefit of priority from U.S. Provisional Application No. 60/712,657, filed Aug. 30, 2005, entitled “Method and System for Depot Contrast Agent for Perfusion Imaging with Intravenous Administration” and U.S. Provisional Application No. 60/735,517, filed Nov. 11, 2005, entitled “Deposit Contrast Agents and Methods,” the disclosures of which are hereby incorporated by reference herein in their entirety.

### GOVERNMENT SUPPORT

This invention was made with government support under Grants Nos. R01-DK063508, R01-HL074443 and R01-HL07810 awarded by the National Institutes of Health. The government has certain rights in the invention.

### BACKGROUND OF THE INVENTION

Tissue perfusion imaging with contrast-enhanced ultrasound (CEU) is currently performed at clinical ultrasound frequencies by imaging microbubble contrast agents that are freely passing through the microcirculation of a tissue. For this application, a stable blood pool concentration of microbubbles is achieved, microbubbles within the imaging plane are destroyed, and the rate and extent of replenishment of contrast-enhancement are measured.

High-frequency, high-resolution ultrasound is increasingly being used to assess small animals in the laboratory because clinical frequency ultrasound does not have sufficient spatial resolution for imaging small animal models of disease in animals such as in mice, rats, and rabbits. Moreover, clinical systems do not have sufficient spatial resolution to image fine structures in people.

There are limitations, however, for contrast perfusion imaging using high frequency ultrasound with available microbubble contrast agents and protocols. These limitations include an inability to destroy microbubbles, a low overall flow signal produced by microbubble agents at high transmit frequency and marked attenuation of acoustic energy from bubbles in the blood pool, particularly the ventricular cavities that makes imaging of the entire heart very difficult. Needed in the art are contrast agent compositions and methods for enhanced ultrasound imaging, including tissue perfusion imaging, for use with high frequency ultrasound systems.

### SUMMARY OF THE INVENTION

A method for generating an enhanced ultrasound image comprises intravenously administering to a subject a plurality of microbubbles of sufficient diameter to lodge in the microvasculature of the subject. An ultrasound image of a portion of the subject is generated wherein the image is enhanced by one or more of the administered microbubbles that has lodged in the microvasculature of the imaged portion.

An ultrasound contrast media composition comprises a plurality of gas filled microbubbles. In one aspect, at least about 5% of the microbubbles have a diameter of at least about 4 microns (μm) and the composition is suitable for intravenous administration. The microbubbles are of sufficient diameter to lodge in the microvasculature of a subject and can be used to enhance ultrasound images from small animal subjects including mice, rats and rabbits.

An aspect of an embodiment of the present invention provides a method of approximating a concentration of microbubbles lodged in the microcirculation of a subject or a portion thereof. The method comprising: intravenously administering a plurality of microbubbles of sufficient diameter to lodge in the microvasculature of the subject; generating an ultrasound image of a portion of the subject wherein the image is enhanced by one or more of the administered microbubbles that has lodged in the microvasculature of the imaged portion; and approximating the concentration of the lodged microbubbles in the imaged portion using the enhanced ultrasound image.

An aspect of an embodiment of the present invention provides a method for evaluating perfusion of blood into tissue of a subject or a portion thereof. The method comprising: intravenously administering a plurality of microbubbles of sufficient diameter to lodge in the microvasculature of the subject; generating an ultrasound image of a portion of the subject wherein the image is enhanced by one or more of the administered microbubbles that has lodged in the microvasculature of the imaged portion; and evaluating perfusion of blood into the tissue of the subject or a portion thereof by approximating the concentration of the lodged microbubbles in the imaged portion using the enhanced ultrasound image.

An aspect of an embodiment of the present invention provides a method for evaluating perfusion of blood into tissue of a subject or a portion thereof. The method comprising: intravenously administering a first dosage comprising a plurality of microbubbles of sufficient diameter to lodge in the microvasculature of the subject; generating a first ultrasound image of a portion of the subject wherein the image is enhanced by one or more of the administered microbubbles that has lodged in the microvasculature of the first imaged portion; approximating a first concentration of the lodged microbubbles in the first imaged portion using the first ultrasound image; disrupting the lodged microbubbles or a portion thereof; administering a pharmacological agent to the subject; intravenously administering a second dosage comprising a plurality of microbubbles of sufficient diameter to lodge in the microvasculature of the subject, generating a second ultrasound image of a portion of the subject wherein the image is enhanced by one or more of the administered microbubbles that has lodged in the microvasculature of the second imaged portion; approximating a second concentration of the lodged microbubbles in the second imaged portion using the second ultrasound image; and evaluating the perfusion of blood into the imaged portion by comparing the first approximated concentration and the second approximated concentration.

An aspect of an embodiment of the present invention provides a method for evaluating perfusion of blood into tissue of a subject or a portion thereof. The method comprising: intravenously administering a first dosage comprising a plurality of microbubbles of sufficient diameter to lodge in the microvasculature of the subject; generating a first ultrasound image of a portion of the subject wherein the image is enhanced by one or more of the administered microbubbles that has lodged in the microvasculature of the first imaged portion; disrupting the lodged microbubbles or a portion thereof; administering a pharmacological agent to the subject; intravenously administering a second dosage comprising a plurality of microbubbles of sufficient diameter to lodge in the microvasculature of the subject; generating a second ultrasound image of a portion of the subject wherein the image is enhanced by one or more of the administered microbubbles that has lodged in the microvasculature of the second imaged portion; and evaluating the perfusion of blood into the imaged portion by comparing the first ultrasound image and the second ultrasound image.

An aspect of an embodiment of the present invention provides an ultrasound contrast media composition. The composition comprising: a plurality of gas filled microbubbles, wherein the microbubbles have a mean diameter of at least about 2.5 microns (μm); and wherein the composition is suitable for intravenous administration.

An aspect of an embodiment of the present invention provides an ultrasound contrast media composition. The composition comprising: a plurality of sulfur hexafluoride filled microbubbles having a lipid shell, wherein the microbubbles have a mean diameter of at least about 2.5 microns (μm); and wherein the composition is suitable for intravenous administration.

An aspect of an embodiment of the present invention provides an ultrasound contrast media composition. The composition comprising at least about 3×10⁵ microbubbles having a diameter between about 4 microns (μm) and about 15 microns (μm) per kilogram (kg) body weight of the subject.

An aspect of an embodiment of the present invention provides an ultrasound contrast media composition. The composition comprising at least about 1.0×10⁷ microbubbles having a diameter of about at least 5 microns (μm) per kilogram (kg) body weight of the subject.

The described methods and compositions can be used to, but not limited thereto, enhance ultrasound images produced using high frequency ultrasound.

These and other objects, along with advantages and features of the invention disclosed herein, will be made more apparent from the description, drawings and claims that follow.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows a short axis CEU image of a mouse heart during transthoracic imaging at 30 MHz 10 s after I.V. injection of 1×10⁷ microbubbles. Opacification of the anterior myocardium can be appreciated but there is severe attenuation (signal dropout) of the rest of the myocardium from microbubbles in the LV cavity.

FIG. 2 shows short axis CEU images of a mouse heart during transthoracic imaging at 30 MHz 10 min after I.V. injection of 1×10⁷ microbubbles demonstrating contrast-enhancement of the entire heart (in the middle panel, opacification in the first frame which is gone after application of a low frequency, high power external energy source). The right panel illustrates increased contrast that can be demonstrated with increase brightness or signal in the grey scale or by background-subtracted color-coded information.

FIG. 3 shows that the mean acoustic intensity was slightly greater for DMPC-DFB microbubbles compared to other agents at both 10% and 50% peak acoustic power.

FIG. 4 shows that the signal enhancement in the anterior LV cavity 10 s after injection was similar for the DSPC-OFP, DSPC-DFB, and DMPC-DFB preparations.

FIG. 5A shows that signal enhancement from microbubbles in the anterior myocardium was not significantly different between agents and was similar when measured at 10 s and 10 min, despite the finding that almost all microbubbles had cleared from the blood pool at the latter interval.

FIG. 5B shows that at 10 s (upper left panel), the high concentration of microbubbles within the LV cavity precluded assessment of myocardial enhancement in any region other than the anterior myocardium whereas at 10 min (upper right panel) all regions could be assessed due to clearance of almost all microbubbles from the cavity. The lower left panel shows that opacification is gone after application of a low frequency, high power external energy source. The opacification can be color coded or shown by an increased brightness or signal in grey scale as shown by the enhanced contrast in the lower right panel.

FIG. 6A shows that myocardial enhancement 10 min after intravenous injection was greatest for the fraction favoring large microbubbles and was the lowest in the fraction favoring small microbubbles.

FIG. 6B shows the degree of delayed enhancement and the relative values for the different populations were not substantially altered when animals were pre-treated with cobra venom factor.

FIG. 7 shows MCE images obtained 10 min after intravenous injection of DSPC-DFB microbubbles in mice with recent LAD infarction.

FIG. 8 shows that a good correlation was found between the two techniques for measurement of the spatial extent of the perfusion defect for each slice and for the summed defect area.

FIG. 9A shows mean (±SD) background-subtracted acoustic intensities for in vitro experiments at 10% and 50% peak acoustic power and, in vivo from the anterior LV cavity 10 s after intravenous injection of microbubbles at 100% peak acoustic power. *p<0.01 vs 10% power.

FIG. 9B shows an image illustrating marked reduction of microbubble signal at the focal zone (arrow) in an in vitro system by placement of an intervening segment of mouse anterior chest wall (ACW, denoted by the bracket)

FIG. 10 shows myocardial enhancement after microbubble injection. FIG. 10A shows examples of MCE images in the mid-ventricular short-axis plane from a mouse obtained after bolus intravenous injection of microbubbles. Images were obtained 10 seconds after injection (upper left), and at 10 min before and after several frames of low-frequency high-power ultrasound to destroy microbubbles. Opacification can be color coded or shown by an increased brightness or signal in grey scale as shown by the enhanced contrast. The background-subtracted image was produced from several pre- and post-destruction frames at 10 min and shows increased brightness in a grey scale. AM stands for anterior myocardium. FIG. 10B shows mean (±SD) background-subtracted acoustic intensities from the anterior myocardium at 10 seconds and 10 minutes. FIG. 10C shows mean (±SD) acoustic intensity from the anterior myocardium at 10 min from the first end-systolic frame (T₀) and at end-systole from 4 subsequent cardiac cycles (T₁-T₄). Data are normalized to T₀.

FIG. 11 shows images and data from a mouse where images were acquired at baseline (BL), 10 s, and at 1 min intervals after intravenous microbubble injection. The final image was acquired after application of low-frequency high-power ultrasound to destroy microbubbles.

FIG. 12 shows mean (±SD) background subtracted acoustic intensity from the anterior myocardium 10 min after injection of size-segregated (small or large populations) microbubbles or the original preparation with mixed size distribution when performed in (A) normal mice or (B) complement-depleted mice.

FIG. 13 shows intravital microscopy data indicating size-related microvsacular retention of microbubbles. FIG. 13A shows the difference between microbubble diameter and capillary diameter according to static microbubble size. Data above the dashed line indicates larger diameter for microbubbles versus vessels. FIG. 13B shows 2 separate static microbubble events. The pseudocolorized images of FITC-labeled vessels and DiI-labeled microbubbles are shown in grey scale and were produced by superimposition of individual images with separate fluorescent filters for microbubbles (left) and vessels (middle). The grey ball shapes indicate lodged microbubbles and arrowheads indicate an example of distal capillary photobleaching that occurred over time due to lack of plasma flux beyond the microbubble. Size bar=20 mm.

FIG. 14A shows images on the left illustrating MCE 10 min after intravenous microbubble injection in successive short-axis planes moving from the base (top) and at 1 mm increments towards to the apex, and corresponding microscopy images of fluorescent nanosphere distribution.

FIG. 14B depicts the relation between perfusion defect size quantified as a percent of the total LV myocardial area measured by late MCE myocardial enhancement and by fluorescent microscopy of nanosphere distribution.

### DETAILED DESCRIPTION OF THE INVENTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific ultrasound systems, or to particular diagnostic protocols, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a microbubble” includes mixtures of microbubble compounds; reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout this application the following abbreviations may be used: AI=acoustic intensity, AV=arteriovenous, DiI=dioctadecyltetramethyl-indocarbocyanine, FITC=fluorescein isothiocyanate, LAD=left anterior descending coronary artery, MCE=myocardial contrast echocardiography, and RBC=red blood cell.

Methods and contrast agents comprising microbubbles are disclosed that overcome limitations to assessing perfusion with high frequency ultrasound in small animals. The methods comprise detection of microbubbles that are deposited in the microcirculation or microvasculature based on their physical size (entrapment mechanism), and yet can still be administered via an intravenous route of injection. As used herein the terms microbubble, contrast agent, ultrasound contrast agent and the like are used interchangeably, unless the context clearly dictates otherwise.

The conventional approach to perfusion imaging with contrast enhanced ultrasound (CEU) involves the creation of a steady state concentration of microbubbles freely circulating in the blood pool, destruction of microbubbles within the imaging plane by high-power imaging sequence, and subsequent measurement of contrast signal regeneration that occurs as microbubbles replenish the beam volume.

The rate and extent of microbubble replenishment measured from CEU time intensity curves reflects microvascular red blood cell velocity and blood volume, respectively, and the product of the two reflects blood flow. This form of imaging is generally performed with low ultrasound transmission frequencies (1-5 MHz) that produces a high microbubble signal relative to noise, and can destroy micro bubbles so that refill kinetics can be evaluated. These frequencies however, do not have sufficient spatial resolution for imaging in small animal models of disease such as in mice, rats, and rabbits.

High frequency imaging systems have been specifically developed for imaging in small animal models of disease. These systems generally operate at a frequency of 20 MHz or higher. However, high frequency imaging has several disadvantages for contrast perfusion imaging. High frequency ultrasound produces less signal enhancement from conventional ultrasound contrast agents due to physical properties of the microbubbles (size distribution, shell properties, etc.). In order to produce contrast enhancement in tissues during high frequency imaging, large doses of contrast agents are administered. When attempting to image tissues such as the heart, the high concentration of microbubbles in the right and left ventricular activities preclude assessment of perfusion in the myocardium in the far field. Another limitation is that microbubbles cannot be easily destroyed at high frequencies since exaggerated and non-linear oscillation that produces inertial cavitation occurs most readily around the lower, ideal resonant frequencies for microbubbles.

The methods and contrast agents described herein comprise microbubbles that can lodge in tissue according to blood flow and yet administration of the agent can still be accomplished by intravenous rather than intra-arterial or intracardiac injection.

After intravenous injection, most microbubbles larger than the dimension of pulmonary capillaries (approximately 5-6 microns) lodge in the pulmonary circulation. However, because there are arteriovenous shunts in the lung that account for up to 5% of transpulmonary flow, microbubbles larger than capillary dimension can transit it to the systemic circulation.

Provided is a method for generating an enhanced ultrasound image. The method comprises intravenously administering a plurality of microbubbles to a subject. A plurality of the administered microbubbles are of sufficient diameter to lodge in the microvasculature of a subject. An ultrasound image can then be generated of the subject, or a portion thereof. The image can be enhanced by one or more of the administered microbubbles that has lodged in the microvasculature of the imaged portion or imaged subject.

Any received ultrasound signal is intended to be included in the term an “ultrasound image.” The term “ultrasound image” is not intended to imply any particular number of ultrasound lines or frames. Thus, one or more lines or frames of ultrasound data, or any other received ultrasound data, can be enhanced by one or more of the administered microbubbles.

Microvasculature includes the portion of the subject's circulatory system composed of the small vessels, such as the capillaries, arterioles, and venules. Such microvasculature is located throughout the tissues and organs of the subject. Microvasculature can also be referred to herein as microcirculation.

The plurality of microbubbles can be in a physiologically acceptable composition for administration to the subject. Such physiologically acceptable compositions can comprise buffers, diluents, therapeutic or pharmacologic agents, pharmacological carriers, preservatives and others compositions known in the art. Thus, an administered physiologically acceptable composition can comprise a plurality of microbubbles in combination with one or more additional components. Such additional components, can be selected by one skilled in the art based factors including, but not limited to, the type of microbubble used and the desired imaging protocol. Factors related to imaging protocol that can direct selection of a suitable additional component, can include, but are not limited to, administration factors (i.e., for example, location), imaging factors (i.e., for example, duration, delay between administration and imaging, tissue or organ imaged, etc.,) and subject factors (i.e, for example, type of subject imaged).

Administration of contrast imaging agents of the present invention can be carried out in various fashions, such as intravascularly, intralymphatically, parenterally, subcutaneously, intramuscularly, intraperitoneally, interstitially, hyperbarically, orally, or intratumorly using a variety of dosage forms. One preferred route of administration is intravascularly. For intravascular use the contrast agent can be injected intravenously, but may be injected intraarterially as well. The useful dosage to be administered and the mode of administration may vary depending upon the age and weight of the subject, and on the particular imaging application intended. The dosage can be initiated at lower levels and increased until the desired contrast enhancement is achieved.

The contrast agent can be administered in the form of an aqueous suspension such as in water or a saline solution (e.g., phosphate buffered saline). The water can be sterile and the saline solution can be a hypertonic saline solution (e.g., about 0.3 to about 0.5% NaCl), although, if desired, the saline solution may be isotonic. The solution also may be buffered, if desired, to provide a pH range of pH 6.8 to pH 7.4. In addition, dextrose may be included in the media.

The contrast agent provided herein, while not limited to a particular use, can be administered intravenously to a laboratory animal. A laboratory animal includes, but is not limited to, a rodent such as a mouse or a rat. As used herein, the term laboratory animal is also used interchangeably with small animal, small laboratory animal, or subject, which includes mice, rats, cats, dogs, fish, rabbits, guinea pigs, rodents, etc. The term laboratory animal does not denote a particular age or sex. Thus, adult and newborn animals, as well as fetuses (including embryos), whether male or female, are included.

The contrast agent can be administered intravenously to a mouse, rat or rabbit. The intravenous injection can be administered as a single bolus dose, or by repeated injection or continuous infusion. Effective dosages and schedules for administering the compositions may be determined empirically, and making such determinations is within the ordinary skill in the art. The dosage range for the administration of the compositions are those large enough to produce a desired ultrasound imaging effect. Such an effect typically includes an increased return from the contrast agent. Such an increased return or intensity of signal from a contrast agent can be indicated by increased brightness on an ultrasound image.

Exemplary dosing can be based on the body weight of the subject and on composition administered. For example, the physiologically acceptable composition administered to the subject can comprise at least about 1×10⁷ microbubbles having a diameter of about at least 4 microns (μm) per (kg) body weight of the subject. In another example, the physiologically acceptable composition administered to the subject can comprise at least about 1×10⁷ microbubbles having a diameter of at least about 5 microns (μm) per (kg) body weight of the subject. In one aspect, the physiologically acceptable composition administered can comprise between at least about 1.0×10⁷ to 6.0×10⁷ microbubbles having a diameter of about at least 5 microns (μm) per kilogram (kg) body weight of the subject. Thus, by non limiting example, at least about 1×10⁷, 2×10⁷, 3×10⁷ 4×10⁷, 5×10⁷, 6×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸ or more, and ranges between these amounts, of microbubbles having a diameter greater than about 4.0 μm or 5.0 μm can be used. For example, 1×10⁷, 2×10⁷, 3×10⁷ 4×10⁷, 5×10⁷, 6×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸or more of the microbubbles can have a diameter of about 4 μm, 5 μm, 6 μm 7 μm, 8 μm, 9 μm, 10 μm, 11 μm, 12 μm, 13 μm, 14 μm, 15 μm, or more and ranges in between. The above proportion of microbubbles above about 5.0 μm can be administered to a subject in a total dosage of, for example, about 0.3×10⁹ to about 1.0×10⁹ microbubbles. Thus, all bubbles of an administered population may or may not be at least about 4 or 5 μm.

In another example, the physiologically acceptable composition administered to the subject can comprise at least about 3×10⁵ microbubbles having a diameter between about 4 microns (μm) and about 15 microns (μm) per kilogram (kg) body weight of the subject. For example, bubbles having a diameter of about 5 μm, 6 μm, 7 μm, 8 μm, 9 μm, 10 μm, 11 μm, 12 μm, 13 μm, 14 μm and ranges between these sizes can be used. The physiologically acceptable composition administered can comprise at least about 3×10⁷, 3×10⁸, 3×10⁹, 4×10⁵, 4×10⁶, 4×10⁷, 4×10⁸, 4×10⁹, 5×10⁵, 5×10⁶, 5×10⁷, 5×10⁸, 5×10⁹, 6×10⁵, 6×10⁶, 6×10⁷, 6×10⁸, 6×10⁹, 7×10⁵, 7×10⁶, 7×10⁷, 7×10⁸, 7×10⁹, 8×10⁵, 8×10⁶, 8×10⁷, 8×10⁸, 8×10⁹, 9×10⁵, 9×10⁶, 9×10⁷, 9×10⁸, 9×10⁹, or more, or ranges between these amounts, of microbubbles having a diameter between about 4 microns (μm) and about 15 microns (μm) per kilogram (kg) body weight of the subject.

For example, the physiologically acceptable composition administered can comprise at least about 3×10⁵ microbubbles having a diameter between about 4 μm and 5 μm, 4 μm and 6 μm, 4 μm and 7 μm, 4 μm and 8 μm, 4 μm and 9 μm, 4 μm and 10 μm, 4 μm and 11 μm, 4 μm and 12 μm, 4 μm and 13 μm, 4 μm and 14 μm, 5 μm and 6 μm, 5 μm and 7 μm, 5 μm and 8 μm, 5 μm and 9 μm, 5 μm and 10 μm, 5 μm and 11 μm, 5 μm and 12 μm, 5 μm and 13 μm, 5 μm and 14 μm, 6 μm and 7 μm, 6 μm and 8 μm, 6 μm and 9 μm, 6 μm and 10 μm, 6 μm and 11 μm, 6 μm and 12 μm, 6 μm and 13 μm, 6 μm and 14 μm, 7 μm and 8 μm, 7 μm and 9 μm, 7 μm and 10 μm, 7 μm and 11 μm, 7 μm and 12 μm, 7 μm and 13 μm, 7 μm and 14 μm, 8 μm and 9 μm, 8 μm and 10 μm, 8 μm and 11 μm, 8 μm and 12 μm, 8 μm and 13 μm, 8 μm and 14 μm, 9 μm and 10 μm, 9 μm and 11 μm, 9 μm and 12 μm, 9 μm and 13 μm, 9 μm and 14 μm, 10 μm and 11 μm, 10 μm and 12 μm, 10 μm and 13 μm, 10 μm and 14 μm, 11 μm and 12 μm, 11 μm and 13 μm, 11 μm and 14 μm, 12 μm and 13 μm, 12 μm and 14 μm and 13 μm and 14 μm.

The physiologically acceptable composition administered can also comprise at least about 3×10⁶ microbubbles having a diameter between about 4 microns (μm) and about 15 microns (μm) per kilogram (kg) body weight of the subject. Moreover, the physiologically acceptable composition administered can comprise between about 3×10⁵ and about 3×10⁶ microbubbles having a diameter between about 4 microns (μm) and about 15 microns (μm) per kilogram (kg) body weight of the subject. The lower end of the above ranges can also start at 5 microns (μm). Thus, the physiologically acceptable composition administered to the subject can comprise at least about 3×10⁵ microbubbles having a diameter between about 5 microns (μm) and about 15 microns (μm) per kilogram (kg) body weight of the subject. The physiologically acceptable composition administered can also comprise at least about 3×10⁶ microbubbles having a diameter between about 5 microns (μm) and about 15 microns (μm) per kilogram (kg) body weight of the subject. The physiologically acceptable composition administered can also comprise at least about 3×10⁷, 3×10⁸, 3×10⁹, 4×10⁵, 4×10⁶, 4×10⁷, 4×10⁸, 4×10⁹, 5×10⁵, 5×10⁶, 5×10⁷, 5×10⁸, 5×10⁹, 6×10⁵, 6×10⁶, 6×10⁷, 6×10⁸, 6×10⁹, 7×10⁵, 7×10⁶, 7×10⁷, 7×10⁸, 7×10⁹, 8×10⁵, 8×10⁶, 8×10⁷, 8×10⁸, 8×10⁹, 9×10⁵, 9×10⁶, 9×10⁷, 9×10⁸, 9×10⁹, or more, or ranges between these amounts, of microbubbles having a diameter between about 5 microns (μm) and about 15 microns (μm) per kilogram (kg) body weight of the subject. Moreover, the physiologically acceptable composition administered can comprise between about 3×10⁵ and about 3×10⁶ microbubbles having a diameter between about 5 microns (μm) and about 15 microns (μm) per kilogram (kg) body weight of the subject

Generally, the dosage can vary with the ultrasound imaging protocol and the desired imaging characteristics, and can be determined by one skilled in the art. The dosage can be adjusted by the individual researcher. Dosage can vary, and can be administered in one or more dose administrations daily, for one or several days. The ultrasound can be transmitted immediately after administration of contrast agent or at any time interval subsequent to contrast agent administration. Ultrasound imaging can also begin prior to administration, continue throughout the administration process, and continue subsequent to the completion of administration. The imaging can also take place at any discrete time prior to, during or after administration of the contrast agent.

For example, the ultrasound image can be generated more than about 1 minute after administration of the physiologically acceptable composition. Optionally, the ultrasound image can generated more than about 3 minutes after administration of the physiologically acceptable composition or the imaged, for example, between about 5 and about 20 minutes after administration of the physiologically acceptable composition, or between about 7 and about 15 minutes after administration of the physiologically acceptable composition. Moreover, times in between those elaborated throughout can be used. For example, images can be generated more than about 1, 2, 3, 4, 5, 6, 7, 8, 9, 10, 11, 12, 13, 14, 15, 16, 17, 18, 19, 20, or 25 minutes after administration of a physiologically acceptable composition or after administration of a contrast media composition and at ranges in between these times. For example images can be generated between about 6 and 20 minutes, 5 and 15 minutes, 1 and 19 minutes, and any other combination of imaging times. Such time frames can be determined based on factors including, but not limited to, the contrast agent and imaging protocol used.

The imaged portion of the subject can be an organ or portion thereof. For example, the organ can be selected from the group consisting of a heart, a brain, a kidney, and a muscle. One non-limiting example of an organ that can be imaged is a heart. A non-limiting example of a muscle type that can be imaged is a skeletal muscle. For example, muscles of the limbs can be imaged. As would be clear to one skilled in the art, however, other muscle types can also be imaged, including smooth muscle, and cardiac muscle, such as when the heart is imaged. Other organs that can be imaged include, but are not limited to a lung, a brain, a liver and blood. The organs imaged or portions thereof can be that of a mouse, rat, or other small animal. The compositions and methods can also be used to image physiological or pathological processes such as angiogenesis or inflammation.

When intravenously administered, the microbubbles travel through the venous system to the right side of the heart. After passing through the right ventricle, the microbubbles are directed into the pulmonary arteries and into the pulmonary circulation. A portion of the microbubbles that are large enough to lodge in microvasculature are shunted through pulmonary arteriovenous shunts into the larger vessels of the pulmonary venous system and are delivered to the left side of the heart. Thus, a portion of the administered microbubbles large enough to lodge in the pulmonary microvasculature can be shunted around the pulmonary microvasculature and thereby avoid entrapment or lodging therein. Once in the left side of the heart, the shunted microbubbles are directed into systemic circulation for deposit in the microvasculature of down stream organs such as the heart myocardium, kidney, brain, liver and skeletal muscles (or that of any other organ with a capillary perfusion bed). Moreover, the microbubbles can be deposited in the microvasculature of tumors or at sights of angiogenesis, or at a site not having a conventional capillary bed. The microbubbles can also be lodged at sites of inflammation.

The shunted microbubbles can lodge in any tissue, organ, or portion thereof having microvasculature and being downstream of the left ventricle. Thus, intravenously administered microbubbles that have lodged in the microvasculature of the imaged portion can have passed through the left side of the subject's heart prior to lodging therein the microvasculature. The image can be enhanced by contacting one or more lodged microbubble(s) with ultrasound and receiving ultrasound or echoes from the one or more contacted microbubble(s). For example, the received ultrasound or echoes from the one or more contacted microbubble can enhance the image by increasing the brightness of the image. Such an enhancement can be based on the non-linear resonance of one or more contacted microbubble, or on reflection of ultrasound without non-linear resonance of the microbubble.

To obtain the enhanced ultrasound image, the one or more lodged microbubble(s) can be contacted with high frequency ultrasound. For example, the ultrasound can be transmitted into the subject at a frequency of about 20 megahertz (MHz) or greater. Optionally, the ultrasound is transmitted into the subject at a frequency of between about 20 MHz and about 80 MHz. Thus, the ultrasound can be transmitted into the subject at a frequency of about 20 MHz, 30 MHz, 40 MHz, 50 MHz, 60 MHz, 70 MHz, 80 MHz, or higher and at ranges in between these frequencies. For example, the ultrasound can be transmitted into the subject at a frequency of about 100 MHz or higher. Moreover, if desired, the ultrasound can be acquired at a high frame rate. For example, the ultrasound can be acquired at about 10 frames per second (fps), 15 fps, 20 fps, 25 fps, 50 fps, 100 fps, 200 fps or more.

Once transmitted, the ultrasound interacts with the laboratory animal's tissues and the contrast agent. The ultrasound is reflected by structures within the animal and scattered non-linearly or reflected by the contrast agent. Echos resulting from interactions with the animal and contrast agent return to an ultrasound imaging system. After ultrasound is received it is processed to form an image. Ultrasound imaging systems may transmit pulsed energy along a number of different directions, or ultrasonic beams, and thereby receive diagnostic information as a function of both lateral directions across the body and axial distance into the body. This information can be displayed as two dimensional, “B-scan” images. Such a two-dimensional presentation gives a planar view, or “slice” through the body and shows the location and relative orientation of many features and characteristics within the body. Furthermore, by tilting or moving the ultrasonic sensor across the body, a third dimension may be scanned and displayed over time, thereby providing three-dimensional information. Other known modes of ultrasound imaging can also be used with the disclosed methods and compositions.

Alternatively, ultrasound returns may be presented in the form of “M-scan” images, where the ultrasound echoes along a particular beam direction are presented sequentially over time, with the two axes being axial distance versus time. Thus, M-scan displays enable diagnosis of rapidly moving structures, such as heart valves.

Some ultrasound systems may combine both B-scan and M-scan images within the same display.

In one aspect, high frequency pulsed-wave Doppler or color flow imaging may be used. A pulsed wave Doppler (PWD)/high frequency flow imaging system can also be used. Such a system can be modified for use with nonlinear signals. Systems can further be modified to enable nonlinear color flow imaging. Any of these systems can be used in combination with one for B-scan imaging. Any system can also be used in conjunction with filters, attenuators, pre-amplifiers and second filters. Therefore the system can integrate PWD and color flow and also can enable nonlinear PWD in addition to color flow imaging.

Other ultrasound imaging systems may simultaneously present multiple ultrasound information, including B-scan, M-scan, and Doppler image displays, along with other information, such as EKG signals and/or phonograms. A not limiting list of exemplary modes that can be used alone or in combination includes B-mode, M-mode, pulsed wave Doppler mode, power Doppler mode, color flow Doppler mode, RF-mode and 3-D mode, C-mode and A-mode.

Ultrasound images are formed by the analysis and amalgamation of multiple pulse echo events. An image is formed, effectively, by scanning regions within a desired imaging area using individual pulse echo events, referred to as “A-Scans”, or ultrasound “lines.” Each pulse echo event requires a minimum time for the acoustic energy to propagate into the subject and to return to the transducer. The image is completed by “covering” the desired image area with a sufficient number of scan lines, referred to as “painting in” the desired imaging area so that sufficient detail of the subject anatomy can be displayed. The number of and order in which the lines are acquired can be controlled by the ultrasound system, which also converts the raw data acquired into an image. Using a combination of hardware electronics and software instructions in a process called “scan conversion,” or image construction, the ultrasound image obtained is rendered so that a user viewing the display can view the subject being imaged.

Imaging modalities which can be used in accordance with the invention include two- and three-dimensional imaging techniques such as B-mode imaging (for example using the time-varying amplitude of the signal envelope generated from the fundamental frequency of the emitted ultrasound pulse, from sub-harmonics or higher harmonics thereof or from sum or difference frequencies derived from the emitted pulse and such harmonics, images generated from the fundamental frequency or the second harmonic thereof being preferred), color Doppler imaging, Doppler amplitude imaging and combinations of these last two techniques with any of the other modalities described herein.

The desired ultrasound for use with the disclosed compositions and methods can be applied, transmitted and received using an ultrasonic scanning device that can supply an ultrasonic signal of at least about 20 MHz to the highest practical frequency. Any ultrasound system or device capable of operating at 20 MHz or above can be used. One such exemplary device is the VisualSonics™ (Toronto, CA) UBM system model VS40 VEVO™ 660. Another device is the VisualSoincs™ (Toronto, CA) model VEVO™ 770. Another such system can have the following components as described in U.S. patent application Ser. No. 10/683,890, US patent application publication 20040122319, of which are incorporated herein by reference.

Other devices capable of transmitting and receiving ultrasound at the desired frequencies can also be used. For example, ultrasound systems using arrayed transducers can be used. One such exemplary array system, which is incorporated herein by reference for its teaching of a high frequency array ultrasound system, is described in U.S. provisional application titled “HIGH FREQUENCY ARRAY ULTRASOUND SYSTEM” by James Mehi, Ronald E. Daigle, Laurence C. Brasfield, Brian Starkoski, Jerrold Wen, Kai Wen Liu, Lauren S. Pflugrath, F. Stuart Foster, and Desmond Hirson, and filed Nov. 2, 2005 and assigned attorney docket number 22126.0023U1.

If a small animal subject is used, it can be positioned on a heated platform with access to anesthetic equipment. Thus, the methods can be used with platforms and apparatus used in imaging small animals including “rail guide” type platforms with maneuverable probe holder apparatuses. For example, the described systems can be used with multi-rail imaging systems, and with small animal mount assemblies as described in U.S. patent application Ser. No. 10/683,168, entitled “Integrated Multi-Rail Imaging System,” U.S. patent application Ser. No. 10/053,748, entitled “Integrated Multi-Rail Imaging System,” U.S. patent application Ser. No. 10/683,870, now U.S. Pat. No. 6,851,392, issued Feb. 8, 2005, entitled “Small Animal Mount Assembly,” and U.S. patent application Ser. No. 11/053,653, entitled “Small Animal Mount Assembly,” which are incorporated herein by reference.

Small animals can be anesthetized during imaging and vital physiological parameters such as heart rate and temperature can be monitored. Thus, the system can include means for acquiring ECG and temperature signals for processing and display. The system can also display physiological waveforms such as an ECG, respiration or blood pressure waveform.

Also provided is the use of a system for producing an ultrasound image using line-based image reconstruction with the contrast agents and the methods provided herein. One example of such a system may have the following components as described in U.S. patent application Ser. No. 10/736,232, U.S. patent application publication 20040236219, U.S. Pat. No. 7,052,460, which are set forth in part below and are incorporated herein by reference. The system for producing an ultrasound image using line based image reconstruction can provide an ultrasound image having an effective frame rate in excess of 200 frames per second. The system incorporates an ECG based technique that enables accurate depiction of a rapidly moving structure, such as a heart, in a small animal, such as a mouse, rat, rabbit, or other small animal, using ultrasound (and ultrasound biomicroscopy).

A typical contrast agent comprises a thin flexible or rigid shell composed of albumin, lipid or polymer confining a gas such as nitrogen or a perflurocarbon. Other examples of representative gases include air, oxygen, carbon dioxide, hydrogen, nitrous oxide, inert gases, sulpher fluorides, hydrocarbons, and halogenated hydrocarbons. Liposomes or other microbubbles can also be designed to encapsulate gas or a substance capable of forming gas as described in U.S. Pat. No. 5,316,771, of which is hereby incorporated by reference herein. In another embodiment, gas or a composition capable of producing gas can be trapped in a virus, bacteria, or cell to form a microbubble contrast agent. The described ultrasound contrast agents improve contrast by acting as sound wave reflectors due to acoustic differences between the agents and surrounding liquid or by resonating.

A wide variety of materials can be used in preparing microbubble membrane or shell. Any compound or composition that aids in the formation and maintenance of the bubble membrane or shell by forming a layer at the interface between the gas and liquid phases can be used. Sonication can be used for the formation of microbubbles, i.e., through an ultrasound transmitting septum or by penetrating a septum with an ultrasound probe including an ultrasonically vibrating hypodermic needle. Optionally, larger volumes of microbubbles can be prepared by direct probe-type sonicator action on the aqueous medium in which microbubbles are formed in the presence of gas (or gas mixtures) or another high-speed mixing technique, such as blending or milling/mixing. Other techniques such as gas injection (e.g. venturi gas injection), mechanical formation such as through a mechanical high shear 15 valve (or double syringe needle) and two syringes, or an aspirator assembly on a syringe, or simple shaking, may be used. Microbubbles can also be formed through the use of a liquid osmotic agent emulsion supersaturated with a modifier gas at elevated pressure introduced into in a surfactant solution.

Thus, the administered microbubbles can comprise one or more gasses. For example, the gas can be a fluorine containing hydrocarbon gas. Optionally, the gas is selected from the group consisting of decafluorobutane, octafluorobutane, perfluorohexane, and dodecofluoropentane. The gas can also be sulfur hexafluoride or nitrogen. The microbubbles are not limited to these gases, however, and other gases used for ultrasound contrast agents can also be used. In one example, a microbubble or plurality thereof can be a phospholipids-stabilized microbubble preparation. Bubbles of the phospholipid-stabilized preparation can comprise any gas including those described herein. For example, the bubbles of the phospholipids-stabilized preparation can comprise sulfur hexafluoride gas.

Gases that can be used alone or in combination include, for example, air; nitrogen; oxygen; carbon dioxide; hydrogen; an inert gas such as helium, argon, xenon or krypton; a sulphur fluoride such as sulphur hexafluoride, disulphur decafluoride or trifluoromethylsulphur pentafluoride; selenium hexafluoride; an optionally halogenated silane such as methylsilane or dimethylsilane; a low molecular weight hydrocarbon (e.g. containing up to 7 carbon atoms), for example an alkane such as methane, ethane, a propane, a butane or a pentane, a cycloalkane such as cyclopropane, cyclobutane or cyclopentane, an alkene such as ethylene, propene, propadiene or a butene, or an alkyne such as acetylene or propyne; an ether such as dimethyl ether; a ketone; an ester; a halogenated low molecular weight hydrocarbon (e.g. containing up to 7 carbon atoms); or a mixture of any of the foregoing. At least some of the halogen atoms in halogenated gases can be fluorine atoms; thus halogenated hydrocarbon gases may, for example, be selected from bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethylfluoride, 1,1-difluoroethane and perfluorocarbons. Representative perfluorocarbons include perfluoroalkanes such as perfluoromethane, perfluoroethane, perfluoropropanes, perfluorobutanes (e.g. perfluoro-n-butane, optionally in admixture with other isomers such as perfluoro-isobutane), perfluoropentanes, perfluorohexanes or perfluoroheptanes; perfluoroalkenes such as perfluoropropene, perfluorobutenes (e.g. perfluorobut-2-ene), perfluorobutadiene, perfluoropentenes (e.g. perfluoropent-1-ene) or perfluoro-4-methylpent-2-ene; perfluoroalkynes such as perfluorobut-2-yne; and perfluorocycloalkanes such as perfluorocyclobutane, perfluoromethylcyclobutane, perfluorodimethylcyclobutanes, perfluorotrimethylcyclobutanes, perfluorocyclopentane, perfluoromethylcyclopentane, perfluorodimethylcyclopentanes, perfluorocyclohexane, perfluoromethylcyclohexane or perfluorocycloheptane. Other halogenated gases include methyl chloride, fluorinated (e.g. perfluorinated) ketones such as perfluoroacetone and fluorinated (e.g. perfluorinated) ethers such as perfluorodiethyl ether. The use of perfluorinated gases, for example sulphur hexafluoride and perfluorocarbons such as perfluoropropane, perfluorobutanes, perfluoropentanes and perfluorohexanes, can be particularly advantageous in view of the recognized high stability in the bloodstream of microbubbles containing such gases. Other gases with physicochemical characteristics which cause them to form highly stable microbubbles in the bloodstream may likewise be useful.

Thus, one or more gasses can be enclosed in a shell to form a microbubble. The shell can comprise a lipid. Optionally, the shell is a lipid monolayer and the gas is decafluorobutane.

A contrast agent can be modified to achieve a desired volume percentage by a filtering process, such as by microfiltration using a porous membrane. Contrast agents can also be modified by allowing larger bubbles to separate in solution relative to smaller bubbles. For example, contrast agents can be modified by allowing larger bubbles to float higher in solution relative to smaller bubbles. A population of microbubbles of an appropriate size to achieve a desired size distribution can subsequently be selected. Other means are available in the art for separating microbubble sizes and can be adapted to select a microbubble population of bubbles, such as by centrifugation.

The number of microbubbles of differing sizes in a population can be determined, for example, using an optical decorrelation method. The diameter of microbubbles making up given population can also be determined and the number percentage of microbubbles at different sizes can also be determined. For optical decorrelation methods a Malvern™ Zetasizer™ (Malvern Instruments, Malvern, UK) or similar apparatus may be used.

The contrast agents can be produced using protocols known in the art. For example, microbubbles can be prepared by sonication of an aqueous suspension of either dimyrstyl- or distearylphosphatidycholine, and PEG-sterate in a saturated atmosphere of decafluorobutane gas. This process results in the production of decafluorobutane microbubbles with a lipid monolayer shell.

Further provided are methods for approximating a concentration of microbubbles lodged in the microvasculature of a subject or a portion thereof and for evaluating perfusion of blood into tissue of a subject or a portion thereof. The perfusion in units of mL/min/g tissue can be determined in a manner similar to determining quantification of radiolabeled or colored microspheres injected into the left side of a subject's heart. After of radiolabeled or colored microspheres injection into the systemic circulation in the left atrium or left ventricle they flow downstream and lodge in arterioles based on size. The concentration of spheres (measured by colorietric/fluorometric assay or radioactivity) in a tissue of interest will be proportional to blood flow to the tissue. Absolute quantification in units of mL/min/g tissue can be determined by a systemic blood sample withdrawal at a known rate during microsphere injection which establishes a standard.

A method of approximating a concentration of microbubbles lodged in the microvasculature of a subject or a portion thereof comprises intravenously administering a plurality of microbubbles of sufficient diameter to lodge in the microvasculature of a subject. The injected microbubbles can be those described herein, and can be injected as described herein. An ultrasound image can be generated of a portion of the subject. The generated image can be enhanced by one or more of the administered microbubbles that has lodged in the microvasculature of the imaged portion. The concentration of the lodged microbubbles in the imaged portion can then be approximated using the enhanced ultrasound image. As described herein, the microbubbles can be in a physiologically acceptable composition and the subject can be a small animal.

The image generated can be enhanced by contacting one or more lodged microbubble with ultrasound and receiving ultrasound from the one or more contacted microbubble. The received ultrasound from the one or more contacted microbubble can enhance the image by increasing the brightness of the image and the concentration can be approximated from the brightness of the generated ultrasound image of the imaged portion of the subject. To approximate the concentration, the enhanced ultrasound image can be compared to a control ultrasound image. The control image can be an image taken using the same imaging protocol as the enhanced ultrasound image, except that the control image is not enhanced by the deposited ultrasound contrast agent or microbubbles.

As described herein, the ultrasound image can be generated more than about 1 minute after administration of the physiologically acceptable composition. For example, the ultrasound image can generated more than about 3 minutes after administration of the physiologically acceptable composition. Optionally, the ultrasound image is generated between about 5 and about 20 minutes after administration of the physiologically acceptable composition. For example the ultrasound image can be generated between about 7 and about 15 minutes after administration of the physiologically acceptable composition.

A method for evaluating perfusion of blood into tissue of a subject or a portion thereof can comprise intravenously administering a plurality of microbubbles of sufficient diameter to lodge in the microvasculature of the subject. The injected microbubbles can be those described herein, and can be injected as described herein. An ultrasound image can be of a portion of the subject. The image can be enhanced by one or more of the administered microbubbles that has lodged in the microvasculature of the imaged portion. Perfusion of blood into the tissue of the subject or a portion thereof can be evaluated by approximating the concentration of the lodged microbubbles in the imaged portion using the enhanced ultrasound image. As described herein, the microbubbles can be in a physiologically acceptable composition and the subject can be a small animal.

The image generated can be enhanced by contacting one or more lodged microbubble with ultrasound and receiving ultrasound from the one or more contacted microbubble. The received ultrasound from the one or more contacted microbubble can enhance the image by increasing the brightness of the image and the concentration can be approximated from the brightness of the generated ultrasound image of the imaged portion of the subject. To approximate the concentration, the enhanced ultrasound image can be compared to a control ultrasound image. The control image can be an image taken using the same imaging protocol as the enhanced ultrasound image, except that the control image is not enhanced by the deposited ultrasound contrast agent or microbubbles.

As described herein, the ultrasound image can be generated more than about 1 minute after administration of the physiologically acceptable composition. For example, the ultrasound image can generated more than about 3 minutes after administration of the physiologically acceptable composition. Optionally, the ultrasound image is generated between about 5 and about 20 minutes after administration of the physiologically acceptable composition. For example the ultrasound image can be generated between about 7 and about 15 minutes after administration of the physiologically acceptable composition.

A method for evaluating perfusion of blood into tissue of a subject or a portion thereof comprises intravenously administering a first dosage comprising a plurality of microbubbles of sufficient diameter to lodge in the microvasculature of the subject. The injected microbubbles can be those described herein, and can be injected as described herein. A first ultrasound image can be generated of a portion of the subject wherein the image is enhanced by one or more of the administered microbubbles that has lodged in the microvasculature of the first imaged portion. A first concentration of the lodged microbubbles can be approximated in the first imaged portion using the first ultrasound image. The lodged microbubbles or a portion thereof can be disrupted.

The contrast agent or microbubble can, if desired, be disrupted or destroyed by a pulse of ultrasound. The pulse of ultrasound can be produced by the same or a different transducer as the transducer producing the imaging frequency ultrasound. Therefore, the methods contemplate using a plurality of ultrasound probes and frequencies. The microbubbles can be disrupted or popped by the ultrasound energy at a frequency above, at, or below 20 MHz. As used throughout, “disrupted” or “destroyed” means that a microbubble is fragmented, ruptured, or cracked such that gas escapes from the microbubble.

After the lodged microbubbles, or a portion thereof, are disrupted a pharmacological agent can be administered to the subject and a second dosage comprising a plurality of microbubbles of sufficient diameter to lodge in the microvasculature of the subject can be intravenously administered to the subject. A second ultrasound image can be generated of a portion of the subject wherein the image is enhanced by one or more of the administered microbubbles that has lodged in the microvasculature of the second imaged portion and a second concentration can be approximated of the lodged microbubbles in the second imaged portion using the second ultrasound image. The perfusion of blood into the imaged portion can be evaluated by comparing the first approximated concentration and the second approximated concentration.

The first and second dosages of the microbubbles can be in a physiologically acceptable composition, as described herein and the subject can be a small animal. The first and second images can be enhanced by contacting one or more lodged microbubble(s) with ultrasound and receiving ultrasound from the one or more contacted microbubble. The received ultrasound from the one or more contacted microbubble can enhance the images by, for example, increasing the brightness of the images. The first and second concentrations can be approximated from the brightness of the first and second generated ultrasound images of the imaged portions of the subject respectively.

As described herein, the ultrasound images can be generated more than about 1 minute after administration of the physiologically acceptable composition. For example, an ultrasound image can generated more than about 3 minutes after administration of the physiologically acceptable composition. Optionally, an ultrasound image is generated between about 5 and about 20 minutes after administration of the physiologically acceptable composition. For example, an ultrasound image can be generated between about 7 and about 15 minutes after administration of the physiologically acceptable composition.

A method for evaluating perfusion of blood into tissue of a subject or a portion thereof comprises intravenously administering a first dosage comprising a plurality of microbubbles of sufficient diameter to lodge in the microvasculature of the subject. The injected microbubbles can be those described herein, and can be injected as described herein. As described above, the microbubbles can be in a physiologically acceptable composition and the subject can be a small animal. A first ultrasound image can be generated of a portion of the subject wherein the image is enhanced by one or more of the administered microbubbles that has lodged in the microvasculature of the first imaged portion.

As described herein, the lodged microbubbles or a portion thereof can be disrupted. Before during, or after disruption, a pharmacological agent can be administered to the subject and a second dosage comprising a plurality of microbubbles of sufficient diameter to lodge in the microvasculature of the subject can be intravenously administered to the subject. A second ultrasound image can be generated of a portion of the subject wherein the image is enhanced by one or more of the administered microbubbles that has lodged in the microvasculature of the second imaged portion. The perfusion of blood into the imaged portion can be evaluated by comparing the first ultrasound image and the second ultrasound image.

Also as described herein, the images can be enhanced by contacting one or more lodged microbubble with ultrasound and receiving ultrasound from the one or more contacted microbubble. The received ultrasound from the one or more contacted microbubble can enhance the images by increasing the brightness of the images. An increase in the brightness of the second ultrasound image as compared to the brightness of the first ultrasound image can indicate that the administered pharmacological agent increased perfusion of blood to the imaged portion. A decrease in the brightness of the second ultrasound image as compared to the brightness of the first ultrasound image can indicate that the administered pharmacological agent decreased perfusion of blood to the imaged portion. If the brightness of the second ultrasound image is substantially the same as the brightness of the first ultrasound image, it can indicate that the administered pharmacological agent did not alter perfusion of blood to the imaged portion.

As described herein, the ultrasound images can be generated more than about 1 minute after administration of the physiologically acceptable composition. For example, an ultrasound image can generated more than about 3 minutes after administration of the physiologically acceptable composition. Optionally, an ultrasound image is generated between about 5 and about 20 minutes after administration of the physiologically acceptable composition. For example, an ultrasound image can be generated between about 7 and about 15 minutes after administration of the physiologically acceptable composition.

The enhancement can be further augmented by alteration of the microbubble shell charge in order to further enhance the percentage of microbubbles lodging within the microvasculature. The microbubbles can also be used to spatially map flow heterogeneity caused by coronary occlusion. Moreover, absolute flow reserve can be determined by comparing signal intensity at rest to that during adenosine A2a administration.

The described methods can be performed using an ultrasound contrast media. An ultrasound contrast media comprises a plurality of microbubbles. The plurality of microbubbles can be in a physiologically acceptable composition for administration to the subject. Thus, the ultrasound contrast media can comprise a plurality of microbubbles in a physiologically acceptable composition. Exemplary ultrasound contrast media compositions that can be used in the disclosed methods are described herein.

Such an ultrasound contrast media composition can comprise a plurality of gas filled microbubbles, wherein at least about 5% of the microbubbles have a diameter of at least about 4 or 5 microns (μm). Thus, for example, any volume of contrast media composition can have a total bubble population wherein at least 5% of the bubbles in that bubble population are 4 micron bubbles. The composition can be suitable for intravenous administration and a plurality of the microbubbles can be of sufficient diameter to lodge in the microvasculature of a subject. The subject can be a small animal as described herein. The ultrasound contrast media composition can further comprise at least about 3% of the microbubbles having diameter of at least about 4 or 5 microns (μm).

Additional percentages of microbubbles of given size can also comprise the contrast media composition. For example, at least about 5%, 10%, 15%, 20%, 25%, 30%, 35%, 40%, 45% 50% or more of the microbubbles can have a diameter of at least about 4 microns (μm). Moreover, the ultrasound contrast media composition can have a percentage of microbubbles within a range of microbubble sizes. For example, at least about 5%, 10%, 15%, 20%, 25%, 30%, 35%, 40%, 45% 50% or more of the microbubbles can have a diameter of between about 4 microns (μm) and about 15 microns (μm). Moreover, at least about 5%, 10%, 15%, 20%, 25%, 30%, 35%, 40%, 45% 50% or more of the microbubbles can have a diameter of at least about 5 microns (μm). For example, at least about 5%, 10%, 15%, 20%, 25%, 30%, 35%, 40%, 45% 50% or more of the microbubbles can have a diameter of between about 5 microns (μm) and about 15 microns (μm). An exemplary volume of a composition can have 5% of its bubbles between 4 and 10 microns in diameter. Higher percentages of bubbles in a volume of composition having a size between 4 and 10 microns can also be used. Such percentages can be determined based on a desired dosage to be administered to a subject. In some examples, 25% or more of the total bubbles in a given volume have a diameter between 4 and 10 microns. A higher percentage of bubbles in the 4-10 micron range can reduce the total dosage given to the subject to perform the disclosed imaging methods. In other examples, greater than 25%, and up to and including 100% of the bubbles in a volume are between 4 and 10 microns.

The ultrasound media composition can comprise at least about 1×10⁷ microbubbles having a diameter of about at least 4 microns (μm) per (kg) body weight of the subject. In another example, the ultrasound media composition can comprise at least about 1×10⁷ microbubbles having a diameter of about at least 5 microns (μm) per (kg) body weight of the subject. In one aspect, the ultrasound media composition can comprise at least about 1×10⁷ to 6.0×10⁷ microbubbles having a diameter of about at least 5 microns (μm) per kilogram (kg) body weight of the subject. Thus, by non limiting example, at least about 1×10⁷, 2×10⁷, 3×10⁷ 4×10⁷, 5×10⁷, 6×10⁷, 8×10⁷, 9×10⁷, b 1×10⁸, 2×10⁸, 3×10⁸, 4×10⁸, 5×10⁸, 6×10⁸, 7×10⁸, 8×10⁸, 9×10⁸ or more, and ranges between these amounts, of microbubbles having a diameter greater than about 5.0 μm can be used. For example, 1×10⁷, 2×10⁷, 3×10⁷, 4×10⁷, 5×10⁷, 6×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸ or more of the microbubbles can have a diameter of about 4 μm, 5 μm, 6 μm 7 μm, 8 μm, 9 μm, 10 μm, 11 μm, 12 μm, 13 μm, 14 μm, 15 μm, or more and ranges in between. The above proportion of microbubbles above about 5.0 μm can be in an ultrasound media composition having a total dosage of about 0.3×10⁹ to about 1.0×10⁹ microbubbles.

The ultrasound contrast media composition can comprise at least about 3×10⁵ microbubbles having a diameter between about 4 microns (μm) and about 15 microns (μm) per kilogram (kg) body weight of the subject. For example, bubbles having a diameter of about 5 μm, 6 μm, 7 μm, 8 μm, 9 μm, 10 μm, 11 μm, 12 μm, 13 μm, 14 μm and ranges between these sizes can comprise the ultrasound contrast media composition. The ultrasound contrast media composition can comprise at least about 3×10⁷, 3×10⁸, 3×10⁹, 4×10⁵, 4×10⁶, 4×10⁷, 4×10⁸, 4×10⁹, 5×10⁵, 5×10⁶, 5×10⁷, 5×10⁸, 5×10⁹, 6×10⁵, 6×10⁶, 6×10⁷, 6×10⁸, 6×10⁹, 7×10⁵, 7×10⁶, 7×10⁷, 7×10⁸, 7×10⁹, 8×10⁵, 8×10⁶, 8×10⁷, 8×10⁸, 8×10⁹, 9×10⁵, 9×10⁶, 9×10⁷, 9×10⁸, 9×10⁹, or more, or ranges between these amounts, of microbubbles having a diameter between about 4 microns (μm) and about 15 microns (μm) per kilogram (kg) body weight of the subject.

For example, the ultrasound contrast media composition can comprise at least about 3×10⁵ microbubbles having a diameter between about 4μm and 5 μm, 4 μm and 6 μm, 4 μm and 7 μm, 4 μm and 8 μm, 4 μm and 9 μm, 4 μm and 10 μm, 4 μm and 11 μm, 4 μm and 12 μm, 4 μm and 13 μm, 4 μm and 14 μm, 5 μm and 6 μm, 5 μm and 7 μm, 5 μm and 8 μm, 5 μm and 9 μm, 5 μm and 10 μm, 5 μm and 11 μm, 5 μm and 12 μm, 5 μm and 13 μm, 5 μm and 14 μm, 6 μm and 7 μm, 6 μm and 8 μm, 6 μm and 9 μm, 6 μm and 10 μm, 6 μm and 11 μm, 6 μm and 12 μm, 6 μm and 13 μm, 6 μm and 14 μm, 7 μm and 8 μm, 7 μm and 9 μm, 7 μm and 10 μm, 7 μm and 11 μm, 7 μm and 12 μm, 7 μm and 13 μm, 7 μm and 14 μm, 8 μm and 9 μm, 8 μm and 10 μm, 8 μm and 11 μm, 8 μm and 12 μm, 8 μm and 13 μm, 8 μm and 14 μm, 9 μm and 10 μm, 9 μm and 11 μm, 9 μm and 12 μm, 9 μm and 13 μm, 9 μm and 14 μm, 10 μm and 11 μm, 10 μm and 12 μm, 10 μm and 13 μm, 10 μm and 14 μm, 11 μm and 12 μm, 11 μm and 13 μm, 11 μm and 14 μm, 12 μm and 13 μm, 12 μm and 14 μm and 13 μm and 14 μm.

The ultrasound contrast media composition can also comprise at least about 3×10⁶ microbubbles having a diameter between about 4 microns (μM) and about 15 microns (μm) per kilogram (kg) body weight of the subject. Moreover, the ultrasound contrast media composition can comprise between about 3×10⁵ and about 3×10⁶ microbubbles having a diameter between about 4 microns (μm) and about 15 microns (μm) per kilogram (kg) body weight of the subject. The ultrasound contrast media composition can comprise at least about 3×10⁵ microbubbles having a diameter between about 5 microns (μm) and about 15 microns (μm) per kilogram (kg) body weight of the subject. The ultrasound contrast media composition can also comprise at least about 3×10⁶ microbubbles having a diameter between about 5 microns (μm) and about 15 microns (μm) per kilogram (kg) body weight of the subject. Moreover, the ultrasound contrast media composition can comprise between about 3×10⁵ and about 3×10⁶ microbubbles having a diameter between about 5 microns (μm) and about 15 microns (μm) per kilogram (kg) body weight of the subject. The ultrasound contrast media composition can also comprise at least about 3×10⁷, 3×10⁸, 3×10⁹, 4×10⁵, 4×10⁶, 4×10⁷, 4×10⁸, 4×10⁹, 5×10⁵, 5×10⁶, 5×10⁷, 5×10⁸, 5×10⁹, 6×10⁵, 6×10⁶, 6×10⁷, 6×10⁸, 6×10⁹, 7×10⁵, 7×10⁶, 7×10⁷, 7×10⁸, 7×10⁹, 8×10⁵, 8×10⁶, 8×10⁷, 8×10⁸, 8×10⁹, 9×10⁵, 9×10⁶, 9×10⁷, 9×10⁸, 9×10⁹, or more, or ranges between these amounts, of microbubbles having a diameter between about 5 microns (μm) and about 15 microns (μm) per kilogram (kg) body weight of the subject.

An ultrasound contrast media composition can also comprise a plurality of gas filled microbubbles, wherein the microbubbles have a mean diameter of at least about 2.5 microns (μm). The composition can suitable for intravenous administration. The microbubbles can also have a mean diameter of at least about 3.0, 4.0, 5.0, 6.0, 7.0, 10.0 or more microns (μm). For example, an ultrasound contrast media composition comprises a plurality of decafluorobutane or sulfur hexafluoride filled microbubbles having a lipid shell, wherein the microbubbles have a mean diameter of at least about 2.5 microns (μm), and wherein the composition is suitable for intravenous administration.

The microbubbles of the ultrasound media composition can comprise one or more gasses as described herein. For example, the gas can be a fluorine containing hydrocarbon gas. The gas can be selected from the group consisting of decafluorobutane, octafluorobutane, perfluorohexane, and dodecofluoropentane. The gas can also be sulfur hexafluoride or nitrogen. Also as described herein, the ultrasound contrast media composition can comprise one or more gasses enclosed in a shell. The shell can be a lipid monolayer. Optionally, the shell is a lipid monolayer and the gas is decafluorobutane or sulfur hexafluoride. A bubble can also be phospholipid-stabilized and comprise sulfur hexafluoride. The shell can also comprise a peptide. The ultrasound media composition can be made using techniques know by those skilled in the art. For example, one of skill in the art would know how to produce a bubble of a given gas and shell type using known methods. Moreover, as described herein, bubble populations can be selected for a given size using techniques known in the art such as, for example, centrifugation and flotation.

The contrast media compositions can advantageously be employed as delivery agents for bioactive moieties such as therapeutic drugs (i.e. agents having a beneficial effect on a specific disease in a living human or non-human animal). Thus, for example, therapeutic compounds can be located in the microbubble, may be linked to part of an encapsulating wall or matrix, e.g. through covalent or ionic bonds, if desired through a spacer arm, or may be physically mixed into such encapsulating or matrix material. To deliver an agent a microbubble can be disrupted as described herein. For example, when microbubbles are disrupted or destroyed, drugs or genes that are housed within them or bound to their shells can be released to the blood stream are then delivered to tissue by convective forces through the permeabilized microvessels. Moreover, if the agent is linked or otherwise attached to the microbubble, the agent can be delivered without disrupting the microbubble. For example, a lodged microbubble can deliver a therapeutic agent linked to its shell without being disrupted.

The contrast media compositions can be used as vehicles for contrast-enhancing moieties for imaging modalities other than ultrasound, for example, but not limited to X-ray, light imaging, and magnetic resonance imaging.

The microbubbles can also be targeted to bind selectively or specifically to a desired target. Such targeting can be used to augment the lodging affect of the bubbles based on physical size.

The targeted contrast agents used in the methods described can be targeted to a variety of cells, cell types, microvasculature walls, microvasculature wall types, antigens, vascular antigens, microvascular antigens, cellular membrane proteins, organs, markers, tumor markers, angiogenesis markers, blood vessels, thrombus, fibrin, and infective agents. For example, targeted microbubbles can be produced that localize to targets expressed in a subject. Desired targets are generally based on, but not limited to, the molecular signature of various pathologies, organs and/or cells. For example, adhesion molecules such as integrin αᵥβ₃, intercellular adhesion molecule-1 (I-CAM-1), fibrinogen receptor GPIIb/IIIa and VEGF receptors are expressed in regions of angiogenesis, inflammation or thrombus. These molecular signatures can be used to localize high frequency ultrasound contrast agents through the use of targeting molecules, including but not limited to, complementary receptor ligands, targeting ligands, proteins, and fragments thereof. Target cell types include, but are not limited to, endothelial cells, neoplastic cells and blood cells. The methods described herein can, for example, use microbubbles targeted to VEGFR2, I-CAM-1, αᵥβ₃ integrin, αᵥ integrin, fibrinogen receptor GPIIb/IIIa, P-selectin, L-selectin, mucosal vascular adressin cell adhesion molecule-1. Moreover, using methods known in the art, complementary receptor ligands, such as monoclonal antibodies, can be readily produced to target other markers in a subject. For example, antibodies can be produced to bind to tumor marker proteins, organ or cell type specific markers, or infective agent markers. Thus, the targeted contrast agents can be targeted, using antibodies, proteins, fragments thereof, or other ligands, as described herein, to sites of neoplasia, angiogenesis, thrombus, inflammation, infection, as well as to diseased or normal organs or tissues including but not limited to blood, heart, brain, blood vessel, kidney, muscle, lung and liver. Optionally, the targeted markers are proteins and may be extracellular or transmembrane proteins. The targeted markers, including tumor markers, can be the extracellular domain of a protein. The antibodies or fragments thereof designed to target these marker proteins can bind to any portion of the protein. Optionally, the antibodies can bind to the extracellular portion of a protein, for example, a cellular transmembrane protein. Antibodies, proteins, or fragments thereof can be made that specifically or selectively target a desired target molecule using methods known in the art.

Such selective or specific binding can be readily determined using the methods and devices described herein. For example, selective or specific binding can be determined in vivo or in vitro by administering a targeted contrast agent and detecting an increase ultrasound scattering from the contrast agent bound to a desired target. Thus a targeted contrast agent can be compared to a control contrast agent having all the components of the targeted contrast agent except a targeting ligand. By detecting increased resonance or scattering from the targeted contrast agent versus a control contrast agent, the specificity or selectivity of binding can be determined. If an antibody or similar targeting mechanism is used, selective or specific binding to a target can be determined based on standard antigen/epitope/antibody complementary binding relationships. Further, other controls can be used. For example, the specific or selective targeting of the microbubbles can be determined by exposing targeted microbubbles to a control tissue, which includes all the components of the test tissue except for the desired target ligand or epitope. To compare a control sample to a test sample, levels of non-linear resonance can be detected by enhanced ultrasound imaging.

Illustrative targeting mechanisms that can be targeted to particular targets and indicated areas of use for targetable diagnostic and/or therapeutic agents include, but are not limited to, antibodies to: CD34, ICAM-1, ICAM-2, ICAM-3, E-selectin, P-selectin, L-selectin, PECAM, CD18 Integrins, VLA-1, VLA-2, VLA-3, VLA-4, VLA-5, VLA-6, GlyCAM, MAdCAM-1, fibrin, and myosin. These and other targeting molecule molecules are identified and discussed in U.S. Pat. No. 6,264,917, which is incorporated by reference herein generally and specifically for purposes of identifying useful targeting molecule molecules.

Specific or selective targeted contrast agents can be produced by methods known in the art, for example, using the methods described. For example, targeted contrast agents can be prepared as perfluorocarbon or other gas-filled microbubbles with a monoclonal antibody on the shell as a ligand for binding to target ligand in a subject as described in Villanueva et al., “Microbubbles Targeted to Intracellular Adhesion Molecule-1 Bind to Activated Coronary Artery Endothelial Cells,” Circulation (1998) 98: 1-5. For example, perfluorobutane can be dispersed by sonication in an aqueous medium containing phosphatidylcholine, a surfactant, and a phospholipid derivative containing a carboxyl group. The perfluorobutane is encapsulated during sonication by a lipid shell. The carboxylic groups are exposed to an aqueous environment and used for covalent attachment of antibodies to the microbubbles by the following steps. First, unbound lipid dispersed in the aqueous phase is separated from the gas-filled microbubbles by floatation. Second, carboxylic groups on the microbubble shell are activated with 1-ethyl-3-(3-dimethylaminopropyl) carbodimide, and antibody is then covalently attached via its primary amino groups with the formation of amide bonds.

Targeted microbubbles can also be prepared with a biotinylated shell as described in Weller et al., “Modulating Targeted Adhesion of an Ultrasound Contrast Agent to Dysfunctional Endothelium,” Ann. Biomed. Engineering, (2002) 30: 1012-1019. For example, lipid-based perfluorocarbon-filled microbubbles can be prepared with monoclonal antibody on the shell using avidin-biotin bridging chemistry using the following protocol. Perfluorobutane is dispersed by sonication in aqueous saline containing phosphatidyl choline, polyethylene glycol (PEG) stearate, and a biotinylated derivative of phosphatidylethanolamine as described in the art. The sonication results in the formation of perfluorobutane microbubbles coated with a lipid monolayer shell and carrying the biotin label. Antibody conjugation to the shell is achieved via avidin-biotin bridging chemistry. Samples of biotinylated microbubbles are washed in phosphate-buffered saline (PBS) by centrifugation to remove the lipid not incorporated in the microbubble shell. Next, the microbubbles are incubated in a solution (0.1-10 μg/mL) of streptavidin of in PBS. Excess streptavidin is removed by washing with PBS. The microbubbles are then incubated in a solution of biotinylated monoclonal antibody in PBS and washed again. The resultant microbubble have antibody conjugated to the lipid shell via biotin-streptavidin-biotin linkage. In another example, for targeted microbubbles, biotinylated microbubbles can be prepared by sonication of an aqueous dispersion of decafluorobutane gas, distearoylphodphatidylcholine, polyethyleneglycol-(PEG-)state, and distearoyl-phosphatidylethanolamine-PEG-biotin. Microbubbles can then be combined with streptavidin, washed, and combined with biotinylated echistatin.

Targeted microbubbles can also be prepared with an avidinated shell, as is known in the art. In a preferred embodiment, a polymer microbubble can be prepared with an avidinated or streptavidinated shell. For example, a polymer contrast agent comprising a functionalized polyalkylcyanoacrylate can be used as described in patent application PCT/EP01/02802 (of which is hereby incorporated by reference herein). Streptavidin can be bonded to the contrast agent via the functional groups of the functionalized polyalkylcyanoacrylate. In a preferred embodiment, avidinated microbubbles can be used in the methods disclosed herein. When using avidinated microbubbles, a biotinylated antibody or fragment thereof or another biotinylated targeting molecule or fragments thereof can be administered to a subject. For example, a biotinylated targeting ligand such as an antibody, protein or other bioconjugate can be used. Thus, a biotinylated antibody, targeting ligand or molecule, or fragment thereof can bind to a desired target within a subject. Once bound to the desired target, the contrast agent with an avidinated shell can bind to the biotinylated antibody, targeting molecule, or fragment thereof. When bound in this way, high frequency ultrasound energy can be transmitted to the bound contrast agent, which can produce non-linear scattering of the transmitted ultrasound energy. An avidinated contrast agent can also be bound to a biotinylated antibody, targeting ligand or molecule, or fragment thereof prior to administration to the subject.

When using a targeted contrast agent with a biotinylated shell or an avidinated shell a targeting ligand or molecule can be administered to the subject. For example, a biotinylated targeting ligand such as an antibody, protein or other bioconjugate, can be administered to a subject and allowed to accumulate at a target site. A fragment of the targeting ligand or molecule can also be used. For example, the target site can be a portion of the wall of the subject's microvasculature.

When a targeted contrast agent with a biotinylated shell is used, an avidin linker molecule, which attaches to the biotinylated targeting ligand can be administered to the subject. Then, a targeted contrast agent with a biotinylated shell is administered to the subject. The targeted contrast agent binds to the avidin linker molecule, which is bound to the biotinylated targeting ligand, which is itself bound to the desired target. In this way a three step method can be used to target contrast agents to a desired target. The intermediate targeting ligand can bind to all of the desired targets detailed herein as would be clear to one skilled in the art.

Targeted contrast agents or non-targeted contrast agents or microbubbles described herein can also comprise a variety of markers, detectable moieties, or labels. Thus, a microbubble contrast agent equipped with or without a targeting ligand or antibody incorporated into the shell of the microbubble can also include another detectable moiety or label. As used herein, the term “detectable moiety” is intended to mean any suitable label, including, but not limited to, enzymes, fluorophores, biotin, chromophores, radioisotopes, colored particles, electrochemical, chemical-modifying or chemiluminescent moieties. Common fluorescent moieties include: fluorescein, cyanine dyes, coumarins, phycoerythrin, phycobiliproteins, dansyl chloride, Texas Red, and lanthanide complexes. Of course, the derivatives of these compounds which are known to those skilled in the art also are included as common fluorescent moieties.

The detection of the detectable moiety can be direct provided that the detectable moiety is itself detectable, such as, for example, in the case of fluorophores. Alternatively, the detection of the detectable moiety can be indirect. In the latter case, a second moiety reactable with the detectable moiety, itself being directly detectable can be employed. The detectable moiety may be inherent to the molecular probe. For example, the constant region of an antibody can serve as an indirect detectable moiety to which a second antibody having a direct detectable moiety can specifically bind.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLE 1

Microbubbles were prepared with a lipid monolayer shell and perfluorocarbon gas core. Particles for ultrasound contrast enhancement were prepared by sonication of an aqueous suspension of either dimyrstyl- or distearylphosphatidycholine, and PEG-sterate in a saturated atmosphere of decafluorobutane gas. This process resulted in the production of decafluorobutane microbubbles with a lipid monolayer shell. Electrozone counting of the particles with a Coulter Multisizer revealed a broad range in microbubble size with a mean diameter of just under 2 μm and a small fraction of microbubbles with a diameter greater than 5 μm. By flotation separation, a population of relatively large microbubbles (5-15 microns) were separated from smaller bubbles. These microbubbles were of sufficient size that they pass through pulmonary arteriovenous shunts (accounting for up to 3-5% of total pulmonary flow in a small animal), and yet lodge in the coronary or other tissue microcirculation or microvasculature after arrival in the systemic circulation. Moreover, these large bubbles resulted in a relatively higher acoustic signal which is important given the relatively low signal to noise ratio at high frequencies (>10 MHz).

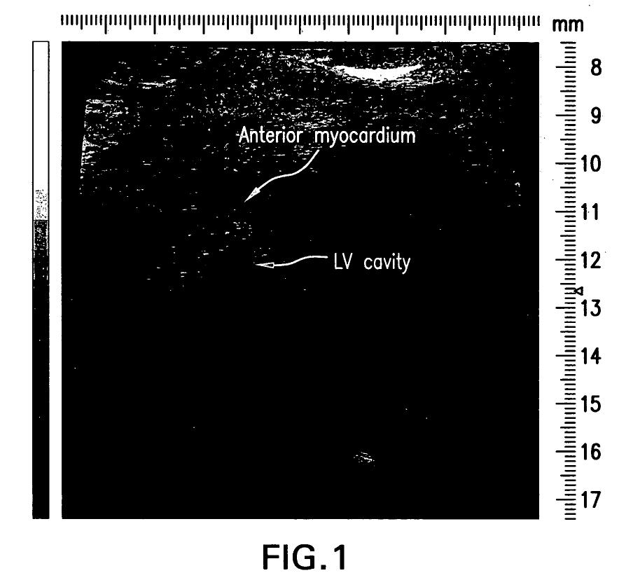

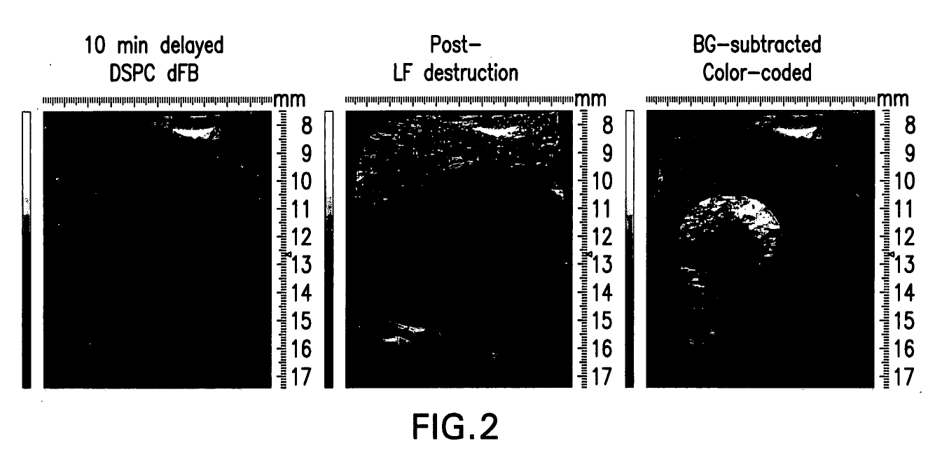

Myocardial imaging at 30 MHz performed immediately after injection of 1×10⁷ of microbubbles demonstrated the presence of contrast effect in the anterior myocardium, but complete shadowing of the all other portions of the heart from attenuation of the ultrasound beam from microbubbles in the LV cavity (FIG. 1). At 10 min, there was a persistent contrast effect detected throughout the myocardium at a time that microbubbles within the blood pool had cleared (indicated by clearance of microbubbles from the ventricular cavities) (FIG. 2). Delayed myocardial contrast effect was further enhanced by 20-30% by flotation separation of microbubbles enriched with population of large microbubbles (approximately 20% of microbubbles >5% μm). This enriched preparation not only augmented lodgining, but produced greater signal-to-noise ratio compared to a standard preparation (by approximately 20%). Modeling of the shunt fraction and known microbubble size distribution indicated that at least 2.5-5.0×10⁴ microbubbles lodge within the murine heart (average weight 150 μmg) after intravenous injection of 1×10⁷ microbubbles.

The method for assessing myocardial perfusion involved a bolus injection of the separated microbubble fraction, then performing delayed enhancement 5-10 min. later after clearance of free agent form the blood pool. Regional or relative blood flow was determined by signal intensity since, similar to radiolabeled or colored microspheres (the laboratory gold standard for flow assessment), the relative concentration of lodged agent is proportional to blood flow per unit mass of tissue.

### EXAMPLE 2

Methods

Microbubble Preparation

Lipid-shelled perfluorocarbon gas microbubble agents were prepared by sonication of a gas-saturated aqueous lipid dispersion. Four separate agents were prepared: distearyl phosphatidylcholine (1.6 mg·mL⁻¹) and PEG-5000 with ocafluoropropane gas (DSPC-OFP); distearyl phosphatidylcholine (1.6 mg·mL⁻¹) and PEG-5000 with decafluorobutane gas (DSPC-DFB); distearyl phosphatidylcholine (1.6 mg·mL⁻¹) and PEG-5000 with sulphur hexafluoride gas (DSPC-SF6); and dimyrystylphosphatidylcholine (1.6 mg·mL⁻¹) and PEG-5000 with decafluorobutane gas (DMPC-DFB). All microbubbles were washed by flotation centrifugation in phosphate-buffered saline (PBS) and their concentration and size distribution were determined by electrozone sensing with a Coulter Multisizer IIe (Beckman-Coulter, Fullerton, Calif.). Separation of microbubbles into small and large size fraction was performed by flotation-centrifugation at 400 g for 15 seconds and separation of the turbid subnatent from the supernatant cake layer which was resuspended in PBS.

In Vivo Measurement of Microbubble Signal

Fundamental ultrasound imaging was performed at 30 MHz with a mechanical sector transducer (Vevo™ 770, VisualSonics, Toronto, CA). Imaging was performed with 2-cycle pulses at acoustic powers of 10% and 50%. The receive gain was optimized for each power and kept constant for all experiments. For each preparation, microbubbles were added to a saline bath containing a magnetic stir bar to achieve a final concentration of 1×10⁵ mL⁻¹. Images were acquired digitally 10 s after injection. Off-line measurements of acoustic intensity (AI) were made (Kulicky, Jabko Inc.) from a region-of-interest spanning 1 mm on either side of the focal zone. Three separate frames were averaged for each measurement and 3 separate measurements were performed for each agent.

Animal Preparation

The study protocol was approved by the Animal Research Committee at the University of Virginia. Male wild-type C57B1/6 mice 8-12 weeks of age were studied. Mice were anesthetized with an intraperitoneal injection (12.5 μL·g⁻¹) of a solution containing ketamine hydrochloride (10 mg·mL⁻¹), xylazine (1 mg·mL⁻¹) and atropine (0.02 mg·mL⁻¹). Body temperature was maintained at 37° C. with a heating platform. A jugular vein was cannulated for administration of microbubbles. Depilatory cream was applied to the anterior and left precordium.

In Vitro Assessment of Blood Pool and Myocardial Microbubble Signal

In 3 mice, contrast echocardiography was performed in a single mid-ventricular short axis plane with the transducer fixed in position to place the focal zone within the LV cavity ⅓ of the distance from the anterior to posterior endocardial surface. The acoustic power was set at 100%. In random order, 1×10⁷ microbubbles for each preparation (non size-separated) was administered as an intravenous bolus injection. After each injection, images were digitally acquired at 10 s and 10 min. Upon completion of image acquisition at 10 min imaging, microbubbles remaining within the myocardium were destroyed by several seconds of continuous low-frequency (1.6 MHz) high-power (mechanical index 1.0-1.2) imaging with phased-array transducer (Sonos 5500, Philips Medical Systems, Bothell, Wash.). High-frequency images were then re-acquired. For each imaging stage, AI from the LV cavity was measured from a region-of-interest placed over the anterior ⅓ of the LV cavity and averaged for 3 separate end-systolic frames. Acoustic intensity was similarly measured from a region-of-interest placed over the anterior myocardium.

To determine whether physical entrapment of microbubbles was responsible for delayed myocardial enhancement that was observed at 10 min, in 6 separate mice imaging studies described above were performed for DSPC-DFB preparations that had been size-segregated by flotation centrifugation. Half of the mice studied were depleted of serum complement by intraperitoneal injection of 10 u of cobra venom factor (Quidel Corp., San Diego, Calif.) divided into equal doses 4 hrs apart starting 18 hrs prior to study in order to assess the contribution of complement-mediated microvascular retention that has been observed for microbubbles with a net negative charge.

Assessment of Intrapulmonary Shunt

In 7 mice, 1×10⁶ fluorophore-labeled (Dye Trak™ VII) polystyrene microspheres (Triton Technology Inc., San Diego, Calif.) with a diameter of 15 μm were administered as an intravenous bolus injection. One minute following injection, mice were euthanized, the heart was immediately removed, and cameral blood was removed by rinsing the heart in PBS. Myocardial tissue was digested in a solution of 1 M KOH and the resulting solution was centrifuged at 1500 g for 15 min. The pellet was resuspended in 10% Triton, centrifuged, then resuspended in 95% acidified EtOH. After a final centrifugation, the pellet was resuspended in PBS (total volume 25 μL) and the total number of spheres present was determined by epi-fluorescent imaging (Axioskop 40, Carl Zeiss Inc., Germany) at an excitation wavelength of 530-560 nm. Assuming that coronary flow represents 7% of cardiac output in mice, the shunt fraction (fₛ) for the 15 μm was calculated by:
fₛ=Nₘ/(Nᵢ·0.07)

where Nₘ is the total number of spheres in the myocardium and Nᵢ is the number of spheres injected.

Assessment of Perfusion by MCE During Myocardial Infarction

In 5 mice, myocardial infarction was produced by ligation of the anterior descending coronary artery. Mice were anesthetized with sodium pentobarbital (100 mg/kg IP) and intubated. Artificial respiration was maintained with a rodent ventilator. After shaving and prepping the anterior chest in a sterile fashion, a parasternal incision spanning the left third and fourth ribs was made. A 7-0 suture was placed around the left anterior descending artery 1-2 mm caudal to the left atrium. The chest was closed in layers and the endotracheal tube was removed once spontaneous breathing resumed.

Myocardial contrast echocardiography was performed 2-3 days following arterial ligation. Ten minutes after an intravenous injection of 1×10⁷ DSPC-DFB microbubbles (non size-separated), images were digitally acquired in the basal short-axis plane. Subsequent short-axis images were acquired after shifting the imaging plane in 1 mm increments in the elevational direction towards the apex by a calibrated stage-positioning micrometer. Images in each plane were re-acquired after several seconds of high-power low-frequency imaging used to destroy microbubbles within the myocardium. A second microbubble injection was performed and imaging was performed at 10 min in a single parasternal long-axis view. Upon completion of imaging, approximately 1×10¹¹ fluorescently-labeled polystyrene spheres (Duke Scientific Corp., Palo Alto, Calif.) with a mean diameter of 500 nm were injected intravenously. The animal was euthanized 20-30 s later and the heart was removed, rinsed in PBS, and the LV was sectioned in the short axis plane in 1 mm increments. Fluorescent epi-illumination (530-560 nm excitation filter) of the apical surface of each myocardial slice was performed with a ×4 objective and digital images were acquired. An image of entire myocardial short axis was produced by reconstruction of individual frames. For each slice, the MCE perfusion defect and region void of fluorescent microspheres were planimetered and defect sizes were expressed as a percentage of the total short-axis LV area. The cumulative defect size was calculated by the summed defect sizes as a percentage of the summed total LV areas.

Statistical Methods

Data are expressed as the mean±SD. Comparisons of continuous variables were made using the students t test (paired) and repeated-measures ANOVA. Correlations were analyzed with regression analysis and data was curve-fitted using a least-squares fit. Differences were considered significant at p<0.05.

Results

In Vitro Microbubble Signal Intensity

The mean microbubble size for the unfractionated samples was similar for the 4 different microbubble preparations (Table 1).

TABLE 1
Size Characteristics for Microbubbles

| Mean diameter | | | | |
|---|---|---|---|---|
| (μm)* | DSPC-OFP | DSPC-DFB | DSPC-SF6 | DMPC-DFB |
| | 1.7 ± 0.2 | 1.5 ± 0.3 | 1.2 ± 0.2 | 1.5 ± 0.5 |

*ANOVA p = ns

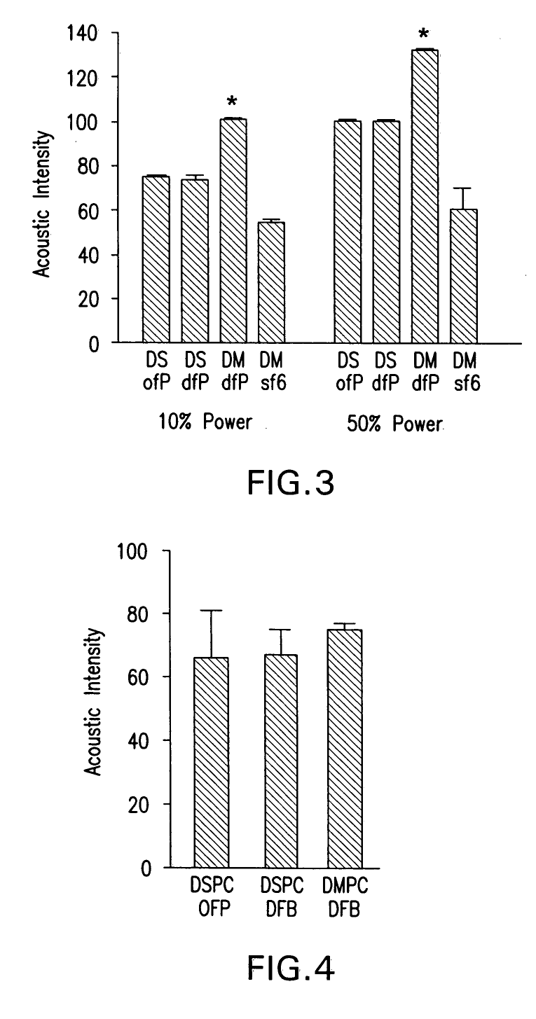

In the in vitro system, the mean acoustic intensity was slightly greater for DMPC-DFB microbubbles compared to other agents at both 10% and 50% peak acoustic power (FIG. 3). The mean signal for DSPC-SF6 tended to be slightly less than that for other agents. This preparation also appeared to be more unstable with a gradual loss of microbubbles over 48-72 hrs after preparation and, hence, was not used for in vivo testing.

Myocardial Contrast Enhancement

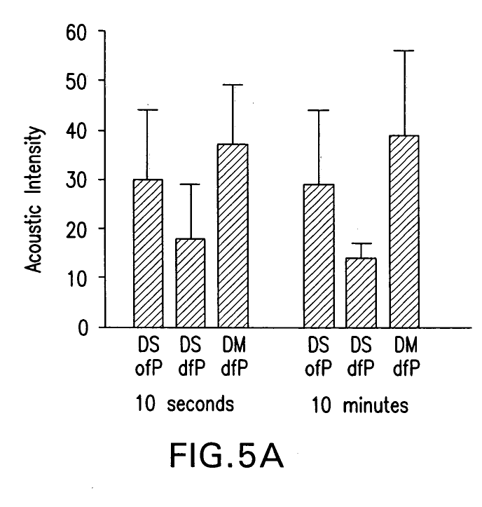

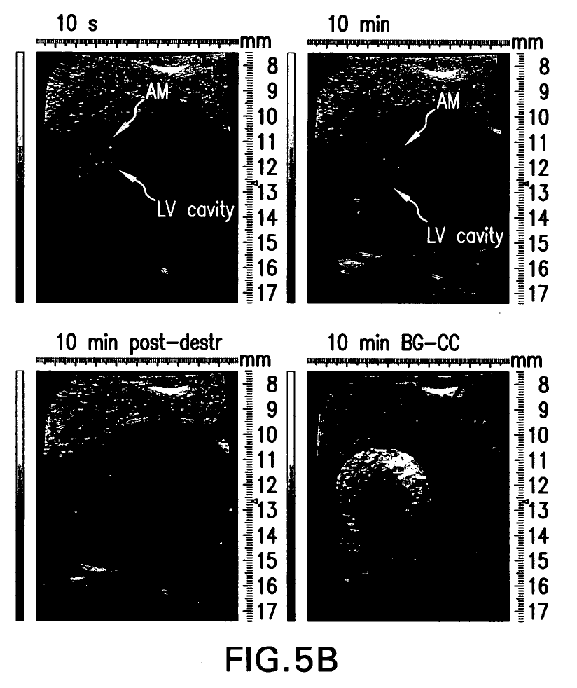

In mice undergoing MCE, the signal enhancement in the anterior LV cavity 10 s after injection was similar for the DSPC-OFP, DSPC-DFB, and DMPC-DFB preparations (FIG. 4). The freely circulating microbubbles had largely cleared from the blood pool by 10 min, demonstrated by a marked decrease in the signal by 10 min. Signal enhancement from microbubbles in the anterior myocardium was not significantly different between agents and was similar when measured at 10 s and 10 min, despite the finding that almost all microbubbles had cleared from the blood pool at the latter interval (FIG. 5A). At 10 s, the high concentration of microbubbles within the LV cavity precluded assessment of myocardial enhancement in any region other than the anterior myocardium whereas at 10 min all regions could be assessed due to clearance of almost all microbubbles from the cavity (FIG. 5B). After the low-frequency high-power imaging sequence at 10 min, myocardial intensity decreased to very low pre-contrast levels. Background-subtraction and color coding demonstrated near-uniform enhancement of the entire left ventricular myocardium with little far field attenuation which can be seen in the increased brightness or signal of the grey scale image shown in the lower right panel of FIG. 5B.

Mechanism for Myocardial Retention of Microbubbles

The persistence of myocardial signal enhancement at a time when freely circulating microbubbles had been largely removed from the blood pool indicated myocardial retention of microbubbles. To determine whether transpulmonary passage and subsequent physical entrapment of microbubbles larger than coronary capillary dimension was responsible for persistent myocardial opacification, the degree of pulmonary shunting was evaluated. Procurement and digestion of hearts after intravenous injection of 15 μm fluorescent microspheres demonstrated 0.1-0.3% of the total administered dose in the myocardium. Based on modeling, these data indicated a pulmonary shunt fraction of 2-4% for particles of this size. To further investigate the mechanism of entrapment, MCE was performed in mice with size-segregated DSPC-DFB preparations. The mean microbubble diameter and percentage >5 μm for each population are presented in Table 2.

TABLE 2
Size Characteristics for DSPC-DFB After Size Separation

| | Original (broad- | Infranatent | Supernatent |
|---|---|---|---|
| | spectrum) | (small) | (large) |
| Mean diameter (μm) | 1.4 ± 0.4 | 2.2 ± 1.2 | 2.7 ± 1.0 |
| Percent > 5 μm (%) | <0.1 | 1.2 | 3.6 |

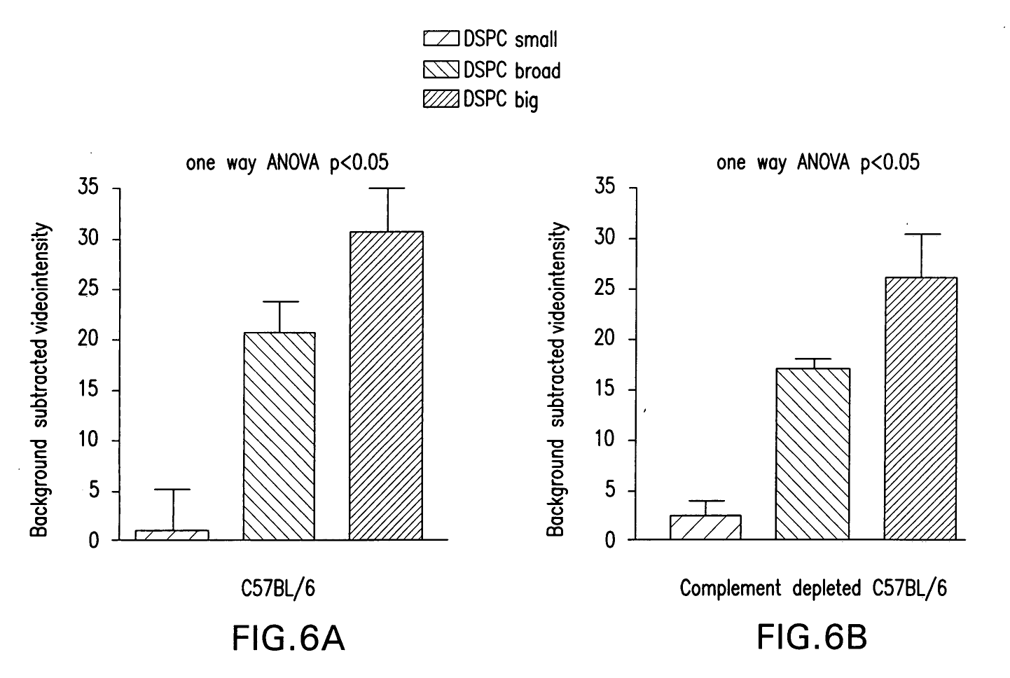

Myocardial enhancement 10 min after intravenous injection was greatest for the fraction favoring large microbubbles and was the lowest in the fraction favoring small microbubbles (FIG. 6A). The degree of delayed enhancement and the relative values for the different populations were not substantially altered when animals were pre-treated with cobra venom factor (FIG. 6B), arguing against complement-mediated microvascular retention as a major mechanism for delayed opacification.

Spatial Assessment of Perfusion in Myocardial Infarction

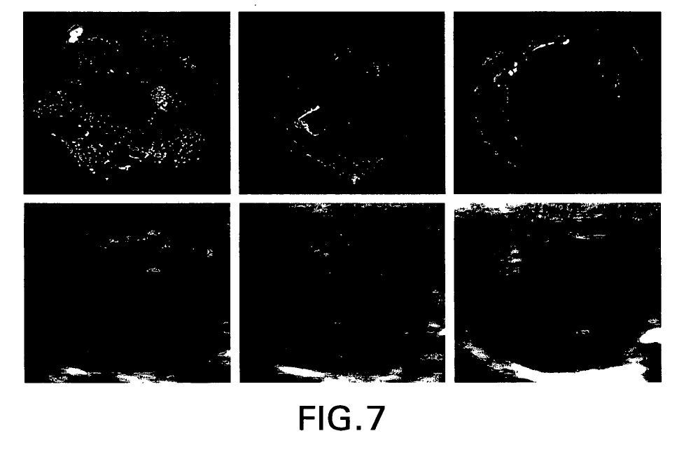

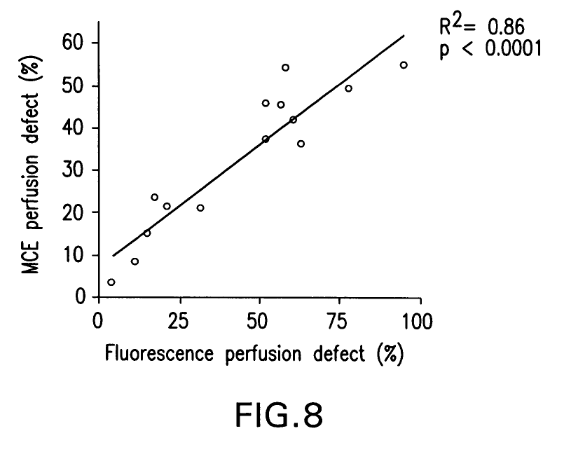

FIG. 7 illustrates MCE images obtained 10 min after intravenous injection of DSPC-DFB microbubbles in mice with recent LAD infarction. For the short axis images, the corresponding reconstructed fluorescent epi-illumination images of nanosphere distribution within the myocardial microcirculation are also shown. Myocardial opacification on MCE correlated spatially with fluorescent microscopy and, when present, transmural differences could be discerned by both techniques. A good correlation was found between the two techniques for measurement of the spatial extent of the perfusion defect for each slice and for the summed defect area (FIG. 8).

### EXAMPLE 3

Contrast Agent Preparation

Lipid-shelled microbubbles were prepared by sonication of an aqueous lipid dispersion of PEG-5000-stearate and distearoyl phosphatidylcholine saturated with decafluorobutane gas. For intravital microscopy studies, the microbubble shell was fluorescently labeled by adding a trace amount of DiI to the suspension prior to sonication. Microbubble concentration and size distribution were determined by electrozone sensing (Coulter Multisizer IIe, Beckman-Coulter, Fullerton, Calif.).

Animal Preparation

Twenty-seven male wild-type C57B1/6 mice 8-12 weeks of age were used. Mice were anesthetized with an intraperitoneal injection (12.5 μL·g⁻¹) of a solution containing ketamine hydrochloride (10 mg·mL⁻¹), xylazine (1 mg·mL⁻¹) and atropine (0.02 mg·mL⁻¹). Body temperature was maintained at 37° C. with a heating platform. A jugular vein was cannulated for administration of microbubbles.

Intravital Micrososcopy

In 4 anesthetized mice, the cremaster muscle was exteriorized and prepared for intravital microscopy during continuous superfusion with isothermic bicarbonate-buffered saline. Observations were made using an Axioskop2-FS microscope (Carl Zeiss, Inc, Thronburg, N.Y.) with a saline-immersion objective (SW 40/0.8 numerical aperture). Video recordings were made with a high-resolution CCD camera (C2400, Hamamatsu Photonics, Hamamatsu, Japan). To image the functional internal diameter of microvessels, 50 mg of FITC-dextran (Mw 70 kD) was injected intravenously followed within 1 min by 1×10⁷ DiI-labeled microbubbles (total volume 100 mL). The muscle preparation was scanned over 5 min. Images of static microbubbles were recorded with fluorescent epi-illumination with excitation filters of 469-500 and 530-560 nm. A second injection of microbubbles was performed >15 min later. Images were digitized and for each static event calibrated video calipers (OsiriX 2.3) were used to measure the capillary diameter in a non-photobleached segment and microbubble diameter perpendicular to the axial direction of the vessel.

Ultrasound

Fundamental ultrasound imaging was performed at 30 MHz with a mechanical sector transducer (Vevo™ 770, Visualsonics, Toronto, Canada). For in vitro protocols, two-cycle pulses were produced at acoustic powers of 10% or 50% of maximal (maximal =PNAP 7.3 MPa, mechanical index 1.4). For MCE, acoustic power was set at 100%. The receive gain was optimized for each power and kept constant for all experiments. For echocardiography, the probe was secured to a railed gantry system and positioned to produce left parasternal short-axis views with the focal zone (approximately 12.5 mm) at the level of the mid-LV cavity. Digital images were transferred to an offline computer for analysis. To assess ultrasound attenuation by the chest wall, in vitro studies were performed in a water tank to measure changes in: a) peak negative and positive pressures by needle hydrophone (PVDF-Z44-0400, Specialty Engineering Associates, Sunnyvale, Calif.); and b) signal intensity from microbubbles (1×10⁵ mL⁻¹) produced by placement of the anterior chest wall from a mouse in the imaging path.

Measurement of Signal Intensity

The ability to produce microbubble-related signal enhancement at 30 MHz was first evaluated in vitro. Microbubbles were suspended in a circulating water bath at a final concentration of 1×10⁵ mL⁻¹ and images at high- or low-power were digitally acquired with constant gain settings. Off-line measurements of AI were made from a region-of-interest spanning 1 mm on either side of the focal plane. Three frames were averaged for each measurement and 3 separate measurements were performed. In vivo measurements were made during MCE in 6 mice. Images were acquired 10 s and 10 min after intravenous bolus injection of 1×10⁶ microbubbles. In 2 of the mice, images were acquired at 1 min intervals over the 10 min period. After the 10 min image acquisition, microbubbles remaining within the myocardium were destroyed by several seconds of exposure to continuous low-frequency (1.6 MHz) high-power (mechanical index 1.0) ultrasound (Sonos 5500, Philips Medical Systems, Bothell, Wash.). Subsequent images at 30 MHz were acquired for background. Background-subtracted acoustic intensity was measured from regions-of-interest placed over the anterior third of the LV cavity and over the anterior myocardium. Data were averaged from 3 separate end-systolic frames.

Evaluation of Size- and Complement-mediated Microbubble Retention

To evaluate whether physical entrapment of microbubbles contributed to persistent myocardial signal enhancement, MCE was performed in 6 additional mice with a standard microbubble preparation (mixed diameter population), a small size population, or a large size population. Separation of microbubbles into small and large size fractions was performed by flotation-centrifugation at 400 g for 15 seconds and separation of the turbid subnatant from the supernatant cake that was resuspended in PBS. Background-subtracted video intensity was determined from the anterior myocardium 10 min after IV injection of 1×10⁶ microbubbles. To investigate the role of complement-mediated retention to the vessel wall, half of the mice studied were depleted of serum complement by intraperitoneal injection of 10 U of cobra venom factor (Quidel Corp., San Diego, Calif.) divided into equal doses 4 hrs apart beginning 18 hrs prior to study.

Assessment of Pulmonary Arterio-venous Shunt Fraction for Microspheres

In 7 mice, 1×10⁶ fluorescent polystyrene microspheres (Triton Technologies, Inc., San Diego, Calif.) with a diameter of 15 μm were administered as an intravenous bolus injection. Two minutes following injection, mice were euthanized, the heart was removed, and blood was removed from the ventricular cavities by rinsing in PBS. Myocardial tissue was digested in a solution of 1 M KOH and was sequentially centrifuged and resuspended in 10% Triton, 95% acidified ethyl alcohol, then PBS (25 μL). The total number of spheres in the myocardium was determined by fluorescent microscopy at an excitation wavelength of 530-560 nm. Assuming that coronary flow represents 7% of cardiac output in mice (10), the shunt fraction (fs) for the 15 μm microspheres was calculated by:
fₛ=Nₘ/(Nᵢ·0.07)

where Nₘ is the total number of spheres in the myocardium and N₁ is the number of spheres injected.

Assessment of Infarct Size by Delayed Opacification

Mice (n=4) were anesthetized, intubated, and ventilated. The anterior chest was prepped in sterile fashion and a parasternal incision spanning the left third and fourth ribs was made. The anterior descending artery was ligated 1-2 mm caudal to the left atrium. The chest was closed in layers and the endotracheal tube was removed. MCE was performed 2-3 days after arterial ligation. Images were acquired 10 min after an intravenous injection of 3×10⁶ microbubbles. A calibrated stage micrometer was used to adjust the imaging plane to acquire short axis images in 1 mm increments from the base to apex. After completion of imaging, 1×10¹¹ fluorescently-labeled polystyrene spheres with a mean diameter of 500 nm (Duke Scientific Corp., Fremont, Calif.) were injected intravenously. The heart was removed 1 min later, rinsed in PBS, and sectioned in the short axis plane in 1 mm increments from the base. Images of the apical surface of each slice were obtained under low magnification fluorescent epi-illumination (530-560 nm excitation filter) and the entire LV short-axis area was reconstructed from individual frames. For each slice, perfusion defect size on MCE and by fluorescent nanosphere distribution were planimetered and expressed as a percentage of the total short-axis LV area. Analysis of perfusion defect sizes were made by a reader blinded to slice identity.

Statistical Methods

Data are expressed as the mean±SD. Comparisons of continuous variables were made using the students t test or repeated measures ANOVA. Pearson correlation was used to analyze association between variables and linear regression with a least-squares fit was used for curve-fitting. Differences were considered significant at p<0.05.

High-frequency Signal Enhancement

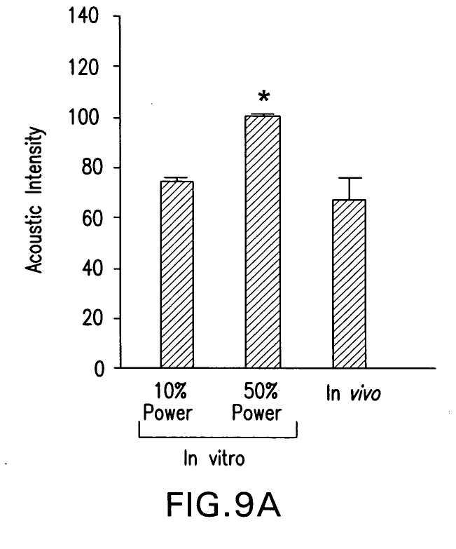

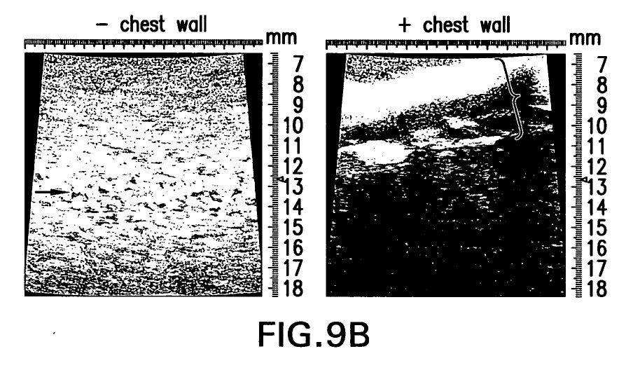

Microbubbles produced signal enhancement during in vitro imaging at 30 MHz, the degree of which was related to acoustic power (FIG. 9A). During MCE, microbubbles produced significant signal enhancement from the anterior LV cavity 10 s after injection (FIG. 9A). According to needle hydrophone measurements, the peak negative and positive acoustic pressures were attenuated by 91% and 89%, respectively, by the mouse anterior chest wall. Microbubble signal enhancement was reduced to a similar degree (88%) by the chest wall (FIG. 9B).

Myocardial Contrast Enhancement

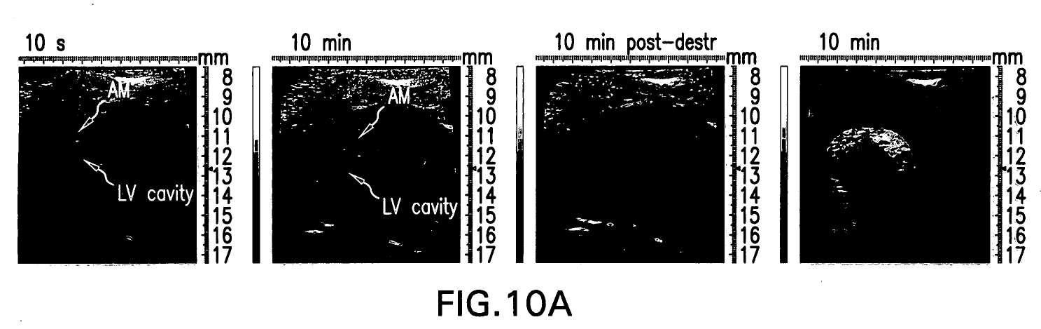

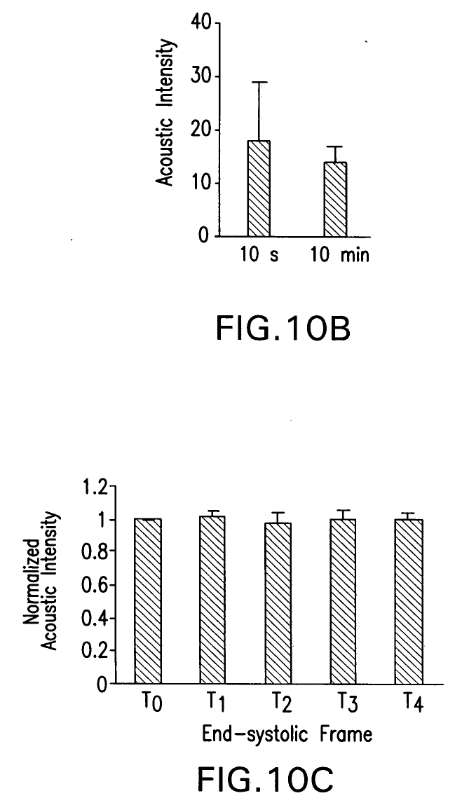

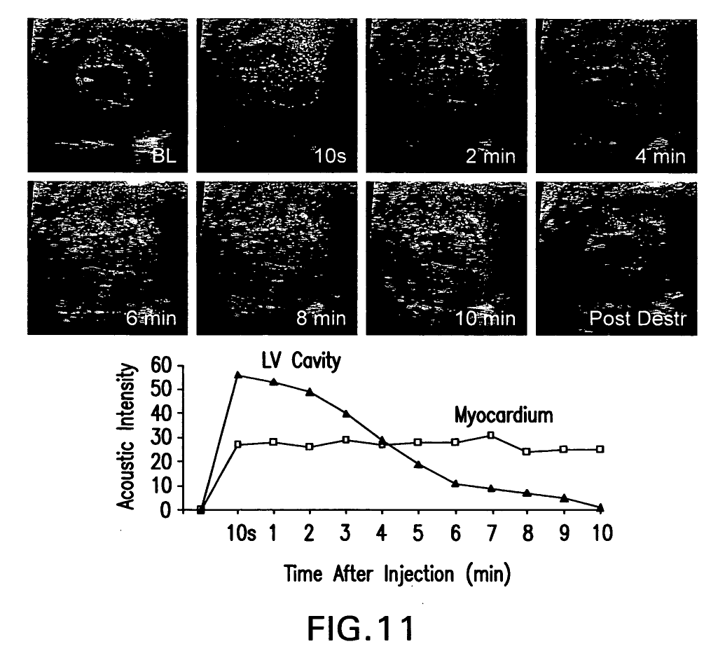

FIG. 10A illustrates MCE short-axis images at 30 MHz after a bolus injection of microbubbles. Early after injection, there was signal enhancement in the anterior myocardium but severe shadowing from LV cavity contrast that precluded evaluation of the posterior segments. Ten minutes after injection most freely-circulating microbubbles had cleared from the blood pool, yet myocardial contrast enhancement persisted. Myocardial video intensity at 10 min retuned to low pre-contrast levels after brief exposure to low-frequency high-power imaging (FIG. 10A). Mean signal enhancement in the myocardium 10 s after injection when microbubble concentration was very high was only slightly greater than that at 10 min when almost all microbubbles had cleared from the blood pool (FIG. 10B). There was little decay of signal enhancement over 5 cardiac cycles after initiation of continuous ultrasound imaging at 10 min indicating that acoustic disruption of microbubbles did not occur (FIG. 10C). In selected mice where signal enhancement was evaluated at 1 min intervals, LV cavity signal rose rapidly after microbubble injection then declined gradually during recirculation phase (FIG. 11). Myocardial signal also rose rapidly and but remained nearly constant from 1 to 10 minutes consistent with first pass retention of agent. By 10 min, the signal from the LV cavity had consistently decreased to a level well below that in the myocardium.

Mechanism for Myocardial Retention of Microbubbles

After intravenous injection of 15 μm fluorescent microspheres, 0.1-0.3% of the total dose lodged in the myocardium, indicating a pulmonary shunt fraction of 2-4%. To further investigate entrapment as a mechanism, MCE was performed with size-segregated microbubble preparations. Table 3 depicts the mean diameter, the percentage of microbubbles with a diameter greater than 5 μm (representing the average diastolic capillary dimension for rat myocardium), and signal enhancement from the blood pool for each of the preparations.

TABLE 3
Size Characteristics and Left Ventricular Cavity Acoustic
Intensity Measurements for Size-segregated Microbubbles

| | Microbubble Population According to Size | | |
|---|---|---|---|
| | Small | Mixed | Large |
| | (Infranatant) | (Original) | (Supernatant) |
| Mean diameter (μm) | 1.4 ± 0.4 | 2.2 ± 1.2 | 2.7 ± 1.0 |
| Fraction > 5 μm (%) | <0.1 | 1.2 | 3.6 |
| LV Cavity Intensity at 10 s | 37 ± 7* | 47 ± 4 | 49 ± 7 |

*p < 0.05 vs mixed and large populations

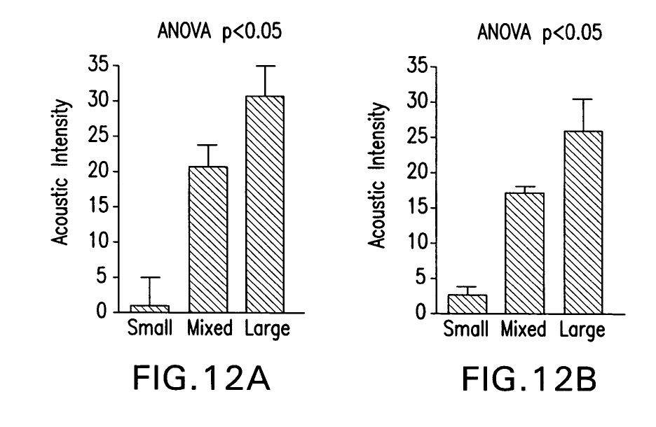

Myocardial signal enhancement 10 min after intravenous injection varied according to the microbubble size distribution (FIG. 12A). Late enhancement was negligible for the small microbubble preparation where <0.1% of the population were greater than 5 μm. The degree of delayed enhancement and the relative values for the different populations were not substantially altered when animals were pre-treated with cobra venom factor (FIG. 12B), indicating that complement-mediated microvascular retention was not a major mechanism for delayed opacification.

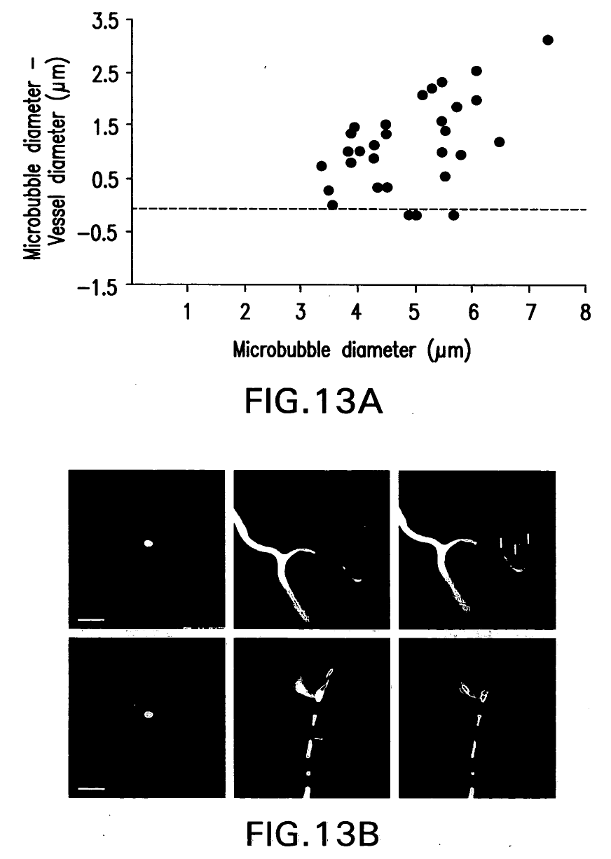

Intravital microscopy was performed to verify microvascular entrapment of microbubbles after their intravenous injection. Static Di-I-labeled microbubbles were observed in capillaries after each injection. Findings consistent with physical entrapment as a mechanism included: (a) a relatively large size for static microbubbles (4.9±1.0 μm) compared to the general population (Table 3); (b) static microbubble diameter larger than that of the adjacent capillary (FIG. 13); (c) lack of plasma flux beyond static microbubbles indicated by preferential photobleaching of FITC-dextran in the distal segment (FIG. 13); and (d) occasional observation of RBC stacking proximal to microbubbles. The only other observed mechanism for microbubble retention was their attachment to adherent leukocytes that occurs in response to muscle exterioration. These events were infrequent and detected only with the second injection which allowed sufficient time to pass for trauma-related leukocyte activation and adhesion.

Spatial Assessment of Perfusion in Myocardial Infarction

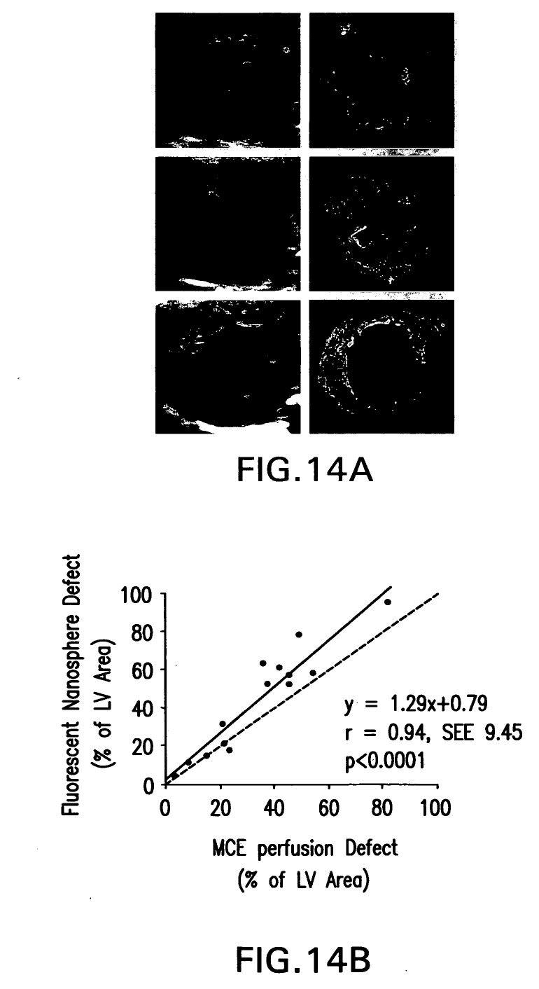

FIG. 14 illustrates MCE images obtained 10 min after intravenous injection of DSPC-DFB microbubbles in mice with recent LAD infarction. For each short-axis image, the corresponding fluorescent epi-illumination image of nanosphere distribution within the myocardial microcirculation are also shown. Myocardial opacification on MCE correlated spatially with fluorescent microscopy and, when present, transmural differences in perfusion was discerned by both techniques. The MCE perfusion defect size was often smaller than the corresponding wall motion defect. A good correlation was found between the two techniques for measurement of the spatial extent of the perfusion defect for each slice (FIG. 14).

Blood Flow Assessment by Depot Tracer Detection

Persistent myocardial opacification was consistently observed late after injection of perfluorocarbon microbubbles with a lipid shell containing PEG. This delayed signal enhancement occurred at a time when the concentration of freely-circulating microbubbles in the blood pool was very low. Agent retention and accumulation resulted in microbubble signal at 10 min almost equal to that measured immediately after a bolus injection when microbubble concentration in the blood pool was very high. Intravital microscopy results confirmed size-related entrapment in capillaries that ranged in diameter from 2.5 to 6 μm in diameter (median 3.7 μm).

Throughout this application, various publications are referenced. The disclosures of these publications (as well as patents, patent applications, and patent application publications) in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A method for generating an enhanced ultrasound image, comprising:
intravenously administering a plurality of microbubbles of sufficient diameter to lodge in the microvasculature of a subject; and
generating an ultrasound image of a portion of the subject wherein the image is enhanced by one or more of the administered microbubbles that has lodged in the microvasculature of the imaged portion.

2. The method of claim 1, wherein the microbubbles are in a physiologically acceptable composition.

3. The method of claim 2, wherein the physiologically acceptable composition administered comprises at least about 3×10⁵ microbubbles having a diameter between about 4 microns (μm) and about 15 microns (μm) per kilogram (kg) body weight of the subject.

4. The method of claim 2, wherein the physiologically acceptable composition administered comprises at least about 3×10⁶ microbubbles having a diameter between about 4 microns (μm) and about 15 microns (μm) per kilogram (kg) body weight of the subject.

5. The method of claim 2, wherein the physiologically acceptable composition administered comprises between about 3×10⁵ and about 3×10⁶ microbubbles having a diameter between about 4 microns (μm) and about 15 microns (μm) per kilogram (kg) body weight of the subject.

6. The method of claim 2, wherein the ultrasound image is generated more than about 1 minute after administration of the physiologically acceptable composition.

7. The method of claim 6, wherein the ultrasound image is generated more than about 3 minutes after administration of the physiologically acceptable composition.

8. The method of claim 7, wherein the ultrasound image is generated between about 5 and about 20 minutes after administration of the physiologically acceptable composition.

9. The method of claim 8, wherein the ultrasound image is generated between about 7 and about 15 minutes after administration of the physiologically acceptable composition.

10. The method of claim 1, wherein the imaged portion of the subject is an organ or portion thereof.

11. The method of claim 10, wherein the organ is selected from the group consisting of a heart, a brain, a kidney, and a muscle.

12. The method of claim 11, wherein the organ is a heart.

13. The method of claim 11, wherein the muscle is a skeletal muscle.

14. The method of claim 1, wherein the microbubbles that lodge in the microvasculature of the imaged portion have passed through the left side of the subject's heart prior to lodging therein the microvasculature.

15. The method of claim 1, wherein ultrasound is transmitted into the subject at a frequency of about 20 megahertz (MHz) or greater.

16. The method of claim 15, wherein the ultrasound is transmitted into the subject at a frequency of between about 20 MHz and about 80 MHz.

17. The method of claim 1, wherein the subject is a small animal.

18. The method of claim 17, wherein the small animal is a rodent.

19. The method of claim 18, wherein the rodent is a mouse.

20. The method of claim 18, wherein the rodent is a rat.

21. The method of claim 17, wherein the small animal is a lagomorph.

22. The method of claim 21, wherein the lagomorph is a rabbit.

23. The method of claim 1, wherein the microbubble comprises one or more gasses.

24. The method of claim 23, wherein the gas is a fluorine containing hydrocarbon gas.

25. The method of claim 24, wherein the gas is selected from the group consisting of decafluorobutane, octafluorobutane, perfluorohexane, and dodecofluoropentane.

26. The method of claim 23, wherein the gas is sulfur hexafluoride or nitrogen.

27. The method of claim 23, wherein the one or more gasses is enclosed in a shell.

28. The method of claim 27, wherein the shell comprises a lipid.

29. The method of claim 28, wherein the shell is a lipid monolayer.

30. The method of claim 29, wherein the shell is a lipid monolayer and the gas is decafluorobutane.

31. The method of claim 27, wherein the shell comprises a peptide.

32. A method of approximating a concentration of microbubbles lodged in the microcirculation of a subject or a portion thereof, comprising:
intravenously administering a plurality of microbubbles of sufficient diameter to lodge in the microvasculature of the subject,
generating an ultrasound image of a portion of the subject wherein the image is enhanced by one or more of the administered microbubbles that has lodged in the microvasculature of the imaged portion; and
approximating the concentration of the lodged microbubbles in the imaged portion using the enhanced ultrasound image.

33. The method of claim 32, wherein the microbubbles are in a physiologically acceptable composition.

34. The method of claim 32, wherein the subject is a small animal.

35. The method of claim 33, wherein the ultrasound image is generated more than about 1 minutes after administration of the physiologically acceptable composition.

36. The method of claim 35, wherein the ultrasound image is generated between about 5 and about 20 minutes after administration of the physiologically acceptable composition.

37. The method of claim 36, wherein the ultrasound image is generated between about 7 and about 15 minutes after administration of the physiologically acceptable composition.

38. A method for evaluating perfusion of blood into tissue of a subject or a portion thereof, comprising:
intravenously administering a plurality of microbubbles of sufficient diameter to lodge in the microvasculature of the subject,
generating an ultrasound image of a portion of the subject wherein the image is enhanced by one or more of the administered microbubbles that has lodged in the microvasculature of the imaged portion; and
evaluating perfusion of blood into the tissue of the subject or a portion thereof by approximating the concentration of the lodged microbubbles in the imaged portion using the enhanced ultrasound image.

39. The method of claim 38, wherein the microbubbles are in a physiologically acceptable composition.

40. The method of claim 38 wherein the subject is a small animal.

41. The method of claim 39, wherein the ultrasound image is generated more than about 1 minute after administration of the physiologically acceptable composition.

42. The method of claim 41, wherein the ultrasound image is generated more than about 3 minutes after administration of the physiologically acceptable composition.

43. The method of claim 42, wherein the ultrasound image is generated between about 5 and about 20 minutes after administration of the physiologically acceptable composition.

44. The method of claim 43, wherein the ultrasound image is generated between about 7 and about 15 minutes after administration of the physiologically acceptable composition.

45. A method for evaluating perfusion of blood into tissue of a subject or a portion thereof, comprising:
intravenously administering a first dosage comprising a plurality of microbubbles of sufficient diameter to lodge in the microvasculature of the subject,
generating a first ultrasound image of a portion of the subject wherein the image is enhanced by one or more of the administered microbubbles that has lodged in the microvasculature of the first imaged portion;
approximating a first concentration of the lodged microbubbles in the first imaged portion using the first ultrasound image;
disrupting the lodged microbubbles or a portion thereof;
administering a pharmacological agent to the subject;
intravenously administering a second dosage comprising a plurality of microbubbles of sufficient diameter to lodge in the microvasculature of the subject,
generating a second ultrasound image of a portion of the subject wherein the image is enhanced by one or more of the administered microbubbles that has lodged in the microvasculature of the second imaged portion;
approximating a second concentration of the lodged microbubbles in the second imaged portion using the second ultrasound image; and
evaluating the perfusion of blood into the imaged portion by comparing the first approximated concentration and the second approximated concentration.

46. The method of claim 45 wherein the first and second dosages of the microbubbles are in a physiologically acceptable composition.

47. The method of claim 45 wherein the subject is a small animal.

48. The method of claim 45, wherein the images are enhanced by contacting one or more lodged microbubble with ultrasound and receiving ultrasound from the one or more contacted microbubble.

49. The method of claim 48, wherein the received ultrasound from the one or more contacted microbubble enhances the images by increasing the brightness of the images.

50. The method of claim 49, wherein the first and second concentrations are approximated from the brightness of the first and second generated ultrasound images of the imaged portions of the subject respectively.

51. The method of claim 46, wherein the ultrasound images are generated more than about 1 minute after administration of the physiologically acceptable composition.

52. The method of claim 51, wherein the ultrasound images are generated more than about 3 minutes after administration of the physiologically acceptable composition.

53. The method of claim 52, wherein the ultrasound images are generated between about 5 and about 20 minutes after administration of the physiologically acceptable composition.

54. The method of claim 53, wherein the ultrasound images are generated between about 7 and about 15 minutes after administration of the physiologically acceptable composition.

55. A method for evaluating perfusion of blood into tissue of a subject or a portion thereof, comprising:
intravenously administering a first dosage comprising a plurality of microbubbles of sufficient diameter to lodge in the microvasculature of the subject,
generating a first ultrasound image of a portion of the subject wherein the image is enhanced by one or more of the administered microbubbles that has lodged in the microvasculature of the first imaged portion;
disrupting the lodged microbubbles or a portion thereof;
administering a pharmacological agent to the subject;
intravenously administering a second dosage comprising a plurality of microbubbles of sufficient diameter to lodge in the microvasculature of the subject,
generating a second ultrasound image of a portion of the subject wherein the image is enhanced by one or more of the administered microbubbles that has lodged in the microvasculature of the second imaged portion; and
evaluating the perfusion of blood into the imaged portion by comparing the first ultrasound image and the second ultrasound image.

56. The method of claim 55, wherein the first and second dosages of the microbubbles are in a physiologically acceptable composition.

57. The method of claim 55, wherein the subject is a small animal.

58. The method of claim 55 wherein the images are enhanced by contacting one or more lodged microbubble with ultrasound and receiving ultrasound from the one or more contacted microbubble.

59. The method of claim 58, wherein the received ultrasound from the one or more contacted microbubble enhances the images by increasing the brightness of the images.

60. The method of claim 59, wherein the an increase in the brightness of the second ultrasound image as compared to the brightness of the first ultrasound image indicates the administered pharmacological agent increased perfusion of blood to the imaged portion.

61. The method of claim 59, wherein the a decrease in the brightness of the second ultrasound image as compared to the brightness of the first ultrasound image indicates the administered pharmacological agent decreased perfusion of blood to the imaged portion.

62. The method of claim 59, wherein the brightness of the second ultrasound image is substantially the same as the brightness of the first ultrasound image indicates the administered pharmacological agent did not alter perfusion of blood to the imaged portion.

63. The method of claim 56, wherein the ultrasound images are generated more than about 1 minute after administration of the physiologically acceptable composition.

64. The method of claim 63, wherein the ultrasound images are generated more than about 3 minutes after administration of the physiologically acceptable composition.

65. The method of claim 64, wherein the ultrasound images are generated between about 5 and about 20 minutes after administration of the physiologically acceptable composition.

66. The method of claim 65, wherein the ultrasound images are generated between about 7 and about 15 minutes after administration of the physiologically acceptable composition.

67. An ultrasound contrast media composition, comprising:
a plurality of gas filled microbubbles, wherein at least about 5% of the microbubbles have a diameter of at least about 4 microns (μm), and wherein the composition is suitable for intravenous administration.

68. The ultrasound contrast media composition of claim 67, wherein the microbubbles are of sufficient diameter to lodge in the microvasculature of a subject.

69. The ultrasound contrast media composition of claim 68, wherein the subject is a small animal.

70. The ultrasound contrast media composition of claim 69, wherein the small animal is a rodent.

71. The ultrasound contrast media composition of claim 70, wherein the rodent is a mouse or a rat.

72. The ultrasound contrast media composition of claim 67, wherein at least about 3% of the microbubbles have diameter of at least about 5 microns (μm).

73. The ultrasound contrast media composition of claim 67, wherein the microbubble comprises one or more gasses.

74. The ultrasound contrast media composition of claim 73, wherein the gas is a fluorine containing hydrocarbon gas.

75. The ultrasound contrast media composition of claim 74, wherein the gas is selected from the group consisting of decafluorobutane, octafluorobutane, perfluorohexane, and dodecofluoropentane.

76. The ultrasound contrast media composition of claim 73, wherein the gas is sulfur hexafluoride or nitrogen.

77. The ultrasound contrast media composition of claim 73, wherein the one or more gasses is enclosed in a shell.

78. The ultrasound contrast media composition of claim 77, wherein the shell comprises a lipid shell.

79. The ultrasound contrast media composition of claim 78, wherein the shell is a lipid monolayer.

80. The ultrasound contrast media composition of claim 79, wherein the shell is a lipid monolayer and the gas is decafluorobutane.

81. The ultrasound contrast media composition of claim 77, wherein the shell comprises a peptide.

82. The ultrasound contrast media composition of claim 67, wherein at least about 10% of the microbubbles have a diameter of at least about 4 microns (μm).

83. The ultrasound contrast media composition of claim 67, wherein at least about 15% of the microbubbles have a diameter of at least about 4 microns (μm).

84. The ultrasound contrast media composition of claim 67, wherein at least about 20% of the microbubbles have a diameter of at least about 4 microns (μm).

85. The ultrasound contrast media composition of claim 67, wherein at least about 25% of the microbubbles have a diameter of at least about 4 microns (μm).

86. The ultrasound contrast media composition of claim 67, wherein at least about 30% of the microbubbles have a diameter of at least about 4 microns (μm).

87. The ultrasound contrast media composition of claim 67, wherein at least about 35% of the microbubbles have a diameter of at least about 4 microns (μm).

88. The ultrasound contrast media composition of claim 67, wherein at least about 5% of the microbubbles have a diameter of between about 4 microns (μm) and about 15 microns (μm).

89. The ultrasound contrast media composition of claim 67, wherein at least about 10% of the microbubbles have a diameter of between about 4 microns (μm) and about 15 microns (μm).

90. The ultrasound contrast media composition of claim 67, wherein at least about 15% of the microbubbles have a diameter of between about 4 microns (μm) and about 15 microns (μm).

91. The ultrasound contrast media composition of claim 67, wherein at least about 20% of the microbubbles have a diameter of between about 4 microns (μm) and about 15 microns (μm).

92. The ultrasound contrast media composition of claim 67, wherein at least about 25% of the microbubbles have a diameter of between about 4 microns (μm) and about 15 microns (μm).

93. The ultrasound contrast media composition of claim 67, wherein at least about 30% of the microbubbles have a diameter of between about 4 microns (μm) and about 15 microns (μm).

94. The ultrasound contrast media composition of claim 67, wherein at least about 35% of the microbubbles have a diameter of between about 4 microns (μm) and about 15 microns (μm).

95. The ultrasound contrast media composition of claim 67, wherein at least about 5% of the microbubbles have a diameter of between about 4 microns (μm) and about 10 microns (μm).

96. The ultrasound contrast media composition of claim 67, wherein at least about 10% of the microbubbles have a diameter of between about 4 microns (μm) and about 10 microns (μm).

97. The ultrasound contrast media composition of claim 67, wherein at least about 15% of the microbubbles have a diameter of between about 4 microns (μm) and about 10 microns (μm).

98. The ultrasound contrast media composition of claim 67, wherein at least about 20% of the microbubbles have a diameter of between about 4 microns (μm) and about 10 microns (μm).

99. The ultrasound contrast media composition of claim 67, wherein at least about 25% of the microbubbles have a diameter of between about 4 microns (μm) and about 10 microns (μm).

100. The ultrasound contrast media composition of claim 67, wherein at least about 30% of the microbubbles have a diameter of between about 4 microns (μm) and about 10 microns (μm).

101. The ultrasound contrast media composition of claim 67, wherein at least about 35% of the microbubbles have a diameter of between about 4 microns (μm) and about 10 microns (μm).

102. An ultrasound contrast media composition, comprising:
a plurality of gas filled microbubbles, wherein the microbubbles have a mean diameter of at least about 2.5 microns (μm), and wherein the composition is suitable for intravenous administration.

103. The ultrasound contrast media composition of claim 102, wherein the microbubble comprises one or more gasses.

104. The ultrasound contrast media composition of claim 103 wherein the gas is a fluorine containing hydrocarbon gas.

105. The ultrasound contrast media composition of claim 104, wherein the gas is selected from the group consisting of decafluorobutane, octafluorobutane, perfluorohexane, and dodecofluoropentane.

106. The ultrasound contrast media composition of claim 103, wherein the gas is sulfur hexafluoride or nitrogen.

107. The ultrasound contrast media composition of claim 103, wherein the one or more gasses is enclosed in a shell.

108. The ultrasound contrast media composition of claim 107, wherein the shell comprises a lipid.

109. The ultrasound contrast media composition of claim 108, wherein the shell is a lipid monolayer.

110. The ultrasound contrast media composition of claim 109, wherein the shell is a lipid monolayer and the gas is decafluorobutane.

111. The ultrasound contrast media composition of claim 107, wherein the shell comprises a peptide.

112. The ultrasound contrast media composition of claim 102, wherein the microbubbles have a mean diameter of at least about 2.5 microns (μm).

113. The ultrasound contrast media composition of claim 102, wherein the microbubbles have a mean diameter of at least about 4.0 microns (μm).

114. The ultrasound contrast media composition of claim 102, wherein the microbubbles have a mean diameter of at least about 5.0 microns (μm).

115. An ultrasound contrast media composition, comprising:
a plurality of sulfur hexafluoride filled microbubbles having a lipid shell, wherein the microbubbles have a mean diameter of at least about 2.5 microns (μm), and wherein the composition is suitable for intravenous administration.

116. An ultrasound contrast media composition, comprising at least about 3×10⁵ microbubbles having a diameter between about 4 microns (μm) and about 15 microns (μm) per kilogram (kg) body weight of the subject.

117. The ultrasound contrast media composition of claim 116, further comprising at least about 3×10⁶ microbubbles having a diameter between about 4 microns (μm) and about 15 microns (μm) per kilogram (kg) body weight of the subject.

118. The ultrasound contrast media composition of claim 116 further comprising between about 3×10⁵ and about 3×10⁶ microbubbles having a diameter between about 4 microns (μm) and about 15 microns (μm) per kilogram (kg) body weight of the subject.

119. The ultrasound contrast media composition of claim 116, wherein the physiologically acceptable composition administered comprises at least about 1.0×10⁵ microbubbles having a diameter of about at least 5 microns (μm) per kilogram (kg) body weight of the subject.

120. The ultrasound contrast media composition of claim 119, wherein the physiologically acceptable composition administered comprises between at least about 1.0×10⁷ and 5.0×10⁷ microbubbles having a diameter of about at least 5 microns (μm) per kilogram (kg) body weight of the subject.

121. An ultrasound contrast media composition, comprising at least about 1.0×10⁷ microbubbles having a diameter of about at least 5 microns (μm) per kilogram (kg) body weight of the subject.

122. The ultrasound contrast media composition of claim 121, comprising between about 1.0×10⁷ and about 5.0×10⁷ microbubbles having a diameter of about at least 5 microns (μm) per kilogram (kg) body weight of the subject.
